(19) United States
(12) Patent Application Publication
(10) Pub. No.: US 2026/0272878 A1
(43) Pub. Date: Sep. 17, 2026

(54) ADAR1 INHIBITORS AND USES THEREOF

(71) Applicant: THE WISTAR INSTITUTE OF ANATOMY AND BIOLOGY, Philadelphia, PA (US)

(72) Inventors: Joseph M. Salvino, Chester Springs, PA (US); Kazuko Nishikura, Haddonfield, NJ (US)

(21) Appl. No.: 19/166,258

(22) PCT Filed: Mar. 18, 2024
(86) PCT No.: PCT/US2024/020403

§ 371 Date: Sep. 17, 2025

Related U.S. Application Data
(60) Provisional application No. 63/490,909, filed on Mar. 17, 2023.

(51) Int. Cl. A61K 31/381; A61K 31/405; A61K 31/4184; A61K 31/52; A61K 39/395; A61P 35/00

(52) U.S. Cl. CPC A61K 31/381; A61K 31/405; A61K 31/4184; A61K 31/52; A61K 39/39558; A61P 35/00

(57) ABSTRACT

Provided herein are ADAR1 inhibitors and methods of treating cancer using the identified compounds.

### STATEMENT REGARDING FEDERALLY SPONSORED RESEARCH OR DEVELOPMENT

This invention was made with government support under grant number CA010815 awarded by the National Institutes of Health. The government has certain rights to the invention.

### BACKGROUND

ADAR (Adenosine Deaminase Acting on RNA) converts adenosine residues to inosine (A-to-I RNA editing) specifically in double-stranded RNA (dsRNA). We identified ADAR1, the first member of the ADAR gene family, which led to identification of ADAR2 and ADAR3. Both ADAR1 and ADAR2 are catalytically active enzymes, whereas no catalytic activity of ADAR3 has been shown so far. A-to-I editing occurs most frequently in non-coding regions that contain repetitive elements Alu and LINE, and many millions of editing sites have been identified in the human transcriptome of these repetitive sequences. Research efforts of our laboratory have been focused on understanding the biological functions of ADAR1. Two ADAR1 isoforms, p150 and p110 (FIG. 21), are generated by the use of separate promoters and alternate splicing. ADAR1p150 is mostly in the cytoplasm, whereas ADAR1p110 mainly localizes in the nucleus.

Despite the remarkable clinical successes of immunotherapy with certain cancers such as melanomas, development of resistance to the therapy in many patients is a major problem. Interestingly, CRISPR screening for factors that regulate this resistance to immune checkpoint blockade unexpectedly identified ADAR1p150 as a major regulator. ADAR1p150 regulates the dsRNA sensing mechanism mediated by melanoma-differentiation associated protein 5 (MDA5), mitochondrial antiviral signaling protein (MAVS), and interferon signaling (MDA5-MAVS-IFN signaling). Hyper-editing of 3′UTR Alu dsRNAs by the cytoplasmic ADAR1p150 desensitizes the dsRNA sensing mechanism and consequently lends tumors resistance to immune checkpoint blockade (FIG. 22), revealing the pro-oncogenic nature of ADAR1. Cancers upregulate the expression of ADAR1 and thereby suppress the MDA5-MAVS-IFN pathway and consequently dampen inflammatory responses, which in turn allows them to escape the PD-1 antibody-initiated immuno-surveillance. Two adenosine analogs, 8-azaadenosine and 8-chloroadenosine, have been claimed as effective ADAR inhibitors, and their anti-proliferative effects on certain cancer cells have been reported by several groups. However, a more rigorous study conducted by Jason Weber's group revealed that these adenosine analogs are in fact not ADAR inhibitors, indicating that anti-cancer activities reported previously must be due to their non-specific cytotoxic activities. No effective ADAR1 inhibitor is currently available, and many pharmaceutical companies are racing to identify an effective inhibitor of ADAR1, which would improve the success rate of PD-1 based immunotherapy.

In contrast to the recent advance in knowledge of ADAR1p150 functions, the biological functions of the nuclear-localized ADAR1p110 have remained mostly unknown.

What is needed is ADAR1 inhibitors and methods of screening for the same.

### SUMMARY OF THE INVENTION

In yet another aspect, a pharmaceutical composition comprising a pharmaceutically acceptable carrier and an ADAR1 inhibitor is provided. The ADAR1 inhibitor includes one or more of:
a) ADARi-13;
b) ADARi-120;
c) ADARi-121;
d) ADARi-122;
e) ADARi-123;
f) ADARi-124;
g) ADARi-125; and
h) ADARi-126,
   or a prodrug, derivative, pharmaceutical salt, or analog thereof.

In certain embodiments, the pharmaceutical composition is a combination product, further comprising a checkpoint inhibitor and/or a chemotherapeutic agent.

In another aspect, a method of treating cancer in a subject is provided. The method includes administering a pharmaceutical composition comprising a pharmaceutically acceptable carrier and an ADAR1 inhibitor is provided. The ADAR1 inhibitor includes one or more of:
a) ADARi-13;
b) ADARi-120;
c) ADARi-121;
d) ADARi-122;
e) ADARi-123;
f) ADARi-124;
g) ADARi-125; and
h) ADARi-126,
   or a prodrug, derivative, pharmaceutical salt, or analog thereof.

In certain embodiments, the pharmaceutical composition is a combination product, further comprising a checkpoint inhibitor and/or a chemotherapeutic agent.

Other aspects and advantages of the invention will be readily apparent from the following detailed description of the invention.

### BRIEF DESCRIPTION OF THE FIGURES

FIG. 1 shows ADAR1p110 together with RNase H2 resolves telomeric R-loops in non-ALT cancer cells by editing A-C mismatches of RNA:DNA hybrids formed between canonical and variant repeats. Telomeric variant repeats cause formation of RNA:DNA hybrids containing A-C mismatches. Unlike RNase H1, RNase H2 cannot degrade the RNA strands of these mismatch-containing RNA:DNA hybrids. Close and specific interaction of ADAR1p110 with RNase H2, but not RNase H1, is detected only in non-ALT cells. In non-ALT cells, upregulated ADAR1p110 edits these A-C mismatches to I:C matched base pairs, which is essential for removal of the RNA strands by RNase H2. The cell cycle specific (G2-M) function of RNase H2 has been recently reported. Thus, the R-loop regulatory function of ADAR1p110 is exerted during G2-M phase. In the absence of ADAR1p110, non-ALT cancer cells die due to genome instability caused by accumulation of telomeric R-loops and mitotic arrest.

FIG. 2A-FIG. 2D show dual luciferase reporter system for measurement of ADAR1 specific A-to-I editing activity and also apoptosis induced in ADAR1 depleted HeLa cells. (FIG. 2A) The dual reporter system was used to monitor ADAR1-mediated A-to-I editing activity. The reporter system contains an A-to-I editing site, downstream of Firefly Luciferase (FFL), at an A-C mismatched base pair within a short hairpin dsRNA. Editing of the adenosine to inosine (equivalent to guanosine) changes an amber stop codon to a tryptophan, resulting in in-frame translation of the downstream Nano Luciferase (Nuc). The ratio of Nuc vs FFL represents efficiency of A-to-I editing of the target site. A reporter HeLa cell line was generated by permanent infection of the reporter Lentivirus. (FIG. 2B) The reporter system is specific for A-to-I editing activity of ADAR1: The reporter activity is reduced only in ADAR1 depleted reporter cells. (FIG. 2C) FFL activity can be used to measure cell viability of reporter HeLa cells. Cytotoxic compounds such as Doxorubicin induces acute apoptosis, which can be detected by reduction of the FFL activity. On the other hand, ADAR1 depletion led to induction of “slow” cell death at 3 to 5 days after siRNA treatment. (FIG. 2D) Induction of slow apoptosis by ADAR1 depletion occurs only in non-ALT (telomerase reactivated) cancer cells due to telomere instability and mitotic arrest. Apoptosis measurement was done using Apo-Tox Glo system 4 days after siRNA treatment.

FIG. 3A-FIG. 3B show identification of ADAR1 specific inhibitors by our own two-step molecular screening strategy. (FIG. 3A) The final two-step screening strategy using the dual luciferase reporter system. Induction of “slow” apoptosis determined as a measure of FFL activity reduction at day 3 was used as the first step screening, resulted in pre-selection of 1,600 compounds. Inhibition of A-to-I editing activity measured as the reduction of Nano Luciferase activity (ratio of Nuc vs FFL) at day 1 was used as the second screening. The absence of extreme and acute cytotoxicity (<40% reduction of FFL at day 1) was also taken into account. (FIG. 3B) Using our well established in vitro A-to-I editing assay, 54 preselected compounds were evaluated for their ADAR1 inhibitory effects using recombinant ADAR1p110 and synthetic dsRNA substrates, known to be targeted by both ADAR1 and ADAR2, as described previously. Although ADAR1 does edit A:U base pairs of completely matched dsRNAs, A-C mismatched base pairs are in fact the favored ADAR1 target sites. Therefore, both matched (top) and mismatched (bottom) dsRNA substrates were tested. Note that some compounds preferentially inhibited matched adenosines, whereas the others had more specific inhibitory effects on adenosine at A-C mismatched base pairs.

FIG. 4 shows determination of IC50 by in vitro editing inhibition assay. In vitro editing assay using purified HAT-ADAR1p110 and a synthetic dsRNA was conducted at varying concentrations of ADAR1i-124. The editing efficiency was determined by the guanosine peak replacing the adenosine peak of cDNA sequencing chromatograms. No ADAR2 inhibition detected.

FIGS. 5A-5D show Biacore SPR analysis for binding of ADAR1i to ADAR1p110. (FIG. 5A) SPR analysis for binding kinetics of 8 compounds to ADAR1p110-WT. ADAR1 inhibition positive (ADAR1i-13, ADAR1i-120, ADAR1i-124) and negative compounds. (FIG. 5B, FIG. 5C) Sonograms of SPR analysis for binding of ADAR1i-13 and ADAR1i-124 to ADAR1p110-WT and ADAR1p110-E912A as well as negative control proteins, GAPDH and HuR, are shown. ADAR1i-13 did not bind to the E912A mutant, while binding kinetics of ADAR1i-124 to the E912A mutant was different from that of wildtype. 5D demonstrates that ADAR1i-124 bound to a mutant ADAR1p110-E912A with binding kinetics different from that of WT. ADAR1-E912, equivalent of ADAR2 E396, is located in the catalytic center. These results indicate that the inhibitor interact with E912 in the catalytic center pocket, perhaps indicating that ADAR1i-124 inhibits ADAR1 mediated editing mechanism by directly binding to the ADAR1 catalytic center.

FIG. 6 shows docking of ADAR1i-124 into the ADAR1 catalytic center pocket generated by αFold. ADAR1i-124 fits well into the active site near Zn²⁺ and makes close contacts with amino acid residues including E912.

FIGS. 7A-7C show analysis of ADAR1i-13 and analogs. (FIG. 7A) Immunostaining using M Ab J2 revealed unedited dsRNA fibers induced in YUMM1.7 cells only by editing inhibition positive compounds (ADAR1i-13, ADAR1i-120, ADAR1i-124). IC50 values determined by dose-dependent in vitro editing assay are shown for ADAR1 inhibition positive compounds. (FIG. 7B) The dsRNA fibers induced by siADAR1 and ADAR1i-124 were sensitive to RNase III treatment. 7C demonstrates that ADAR1i-124 induces formation of Z-RNAs. Dumbbell-shaped dsRNA has been reported to form Z-RNA conformation and act as a strong IFN signaling activation triggers. ADAR1p150 mediated A-to-I editing suppresses Z-RNA structure transition and activation of IFN signaling. We confirmed that ADAR1i-124 induced Z-RNA formation as did siADAR1 RNA.

FIGS. 8A-8C show analysis of ADAR1i-124 effects in cancer cells. (FIG. 8A) ADAR1i-124 (10 μM) selectively reduced viability of HeLa non-ALT cancer cells. Viability was examined using the Apo Tox-Glo assay system. Control, 0.3% DMSO. Data: mean±SD (n=3). Significant differences identified by two-tailed Student's t-tests: *P<0.05, n.s., not significant. (FIG. 8B) Western blotting analysis of HeLa cells treated with ADAR1i-124 (10 μM). Note upregulated g-H2AX, Histone 3 phosphorylated at Serine 10, and cleaved PARP bands. (FIG. 8C) Dot blot analysis for RNA:DNA hybrids induced in HeLa cells by ADAR1i-124 (10 μM).

FIGS. 9A-9C show dose-dependent killing of cancer cells and activation of IFN signaling. (FIG. 9A) Human (HeLa, WM3000) and mouse (YUMM1.7, HSG2) non-ALT cancer cells were dose-dependently killed by ADAR1i-124, while U2OS and IMR90 cells were unaffected. (FIG. 9B, 9C) Activation of the IFN signaling. YUMM1.7 (FIG. 9B) and HGS2 cells (FIG. 9C) were treated with ADAR1i-124 at 10 μM (12 hrs). RNA samples were analyzed by qRT-PCR to monitor ISG expression levels. Data: mean±SD (n=3). Significant differences were identified by two-tailed Student's t-tests: ***P<0.001.

FIGS. 10A-10C show ADAR1i-124 kills cancer cells via different cell death mechanism. (FIG. 10A) Differential expression of ADAR1p150 and ADAR1p110 in cancer cells. (FIG. 10B) HeLa cells were killed via apoptosis, while YUMM1.7 cells killed via necroptosis by siADAR1 and ADAR1i-124. Annexin V and pMLKL were used as apoptosis and necroptosis markers, respectively. At least 200 cells were examined. Data: mean±SD (n=3). Significant differences were identified by two tailed Student's t-tests: *P<0.05, n.s., not significant. FIG. 10C demonstrates ADAR1-124 dose-dependently and efficiently killed mouse melanoma and ovarian cancer cells. We plan to evaluate ADAR1 inhibitors in vivo in mouse tumor model systems by using these mouse cancer cell lines.

FIGS. 11A and 11B demonstrate that ADARi-124 suppresses growth of melanoma in vivo. We inoculated mouse melanoma cells subcutaneously in B6 black mice. Then, starting at Day 8, we treated mice for 4 treatments of either vehicle only or ADAR1i-124 (FIG. 11A). FIG. 11B demonstrates that ADAR1i-124 significantly suppressed growth of mouse melanomas.

### DETAILED DESCRIPTION OF THE INVENTION

Provided herein are ADAR1 inhibitors and methods of using the same for the treatment of telomerase positive cancer. The ADAR1 inhibitors can be used alone or in combination with other cancer therapies, e.g., a checkpoint inhibitor therapy.

### ADAR1

Adenosine deaminase acting on RNA (ADAR) is the enzyme involved in adenosine-to-inosine RNA editing (A-to-I RNA editing), and three ADAR gene family members (ADAR1, ADAR2, and ADAR3) have been identified in vertebrates. ADARs share common domain structures, such as multiple dsRNA-binding domains (dsRBDs) and a separate catalytic domain. Both ADAR1 (ADAR, DRADA) and ADAR2 (ADARB1) are catalytically active enzymes, whereas no catalytic activity of ADAR3 (ADARB2) has been shown so far. A-to-I editing occurs most frequently in noncoding regions that contain repetitive elements Alu and LINE, and many millions of editing sites have been identified in the human transcriptome of these repetitive sequences.

Two ADAR1 isoforms, p150 and p110, are generated by the use of separate promoters and alternate splicing. ADAR1p150 is mostly in the cytoplasm, whereas ADAR1p110 mainly localizes in the nucleus. The cytoplasmic ADAR1p150 regulates the dsRNA sensing mechanism mediated by melanoma-differentiation associated protein 5 (MDA5), mitochondrial antiviral signaling protein (MAVS), and interferon signaling (MDA5-MAVS-IFN signaling). The cytoplasmic ADAR1p150 edits 3′-untranslated region (3′-UTR) dsRNAs primarily comprising inverted Alu repeats and thereby suppresses activation of MDA5-MAVS-IFN signaling. This ADAR1p150 function in the regulation of the MDA5-MAVS-IFN pathway underlies the embryonic lethality of Adar1-null mice and also the pathogenesis of Aicardi-Goutières syndrome (AGS; AGS1-7 subgroups known), a severe human autoimmune disease against endogenous nucleic acids. Mutations of seven genes, including RNaseH2A (AGS4), RNaseH2B (AGS2), RNaseH2C (AGS3), and ADAR1 (AGS6), have been identified in association with AGS, and ten AGS6 mutations of ADAR1 have been reported so far. Finally, this ADAR1p150-mediated suppression of IFN signaling also represses tumor responsiveness to immune checkpoint blockade, revealing the pro-oncogenic ADAR1p150 function. Analysis of The Cancer Genome Atlas database revealed elevated ADAR1 expression and A-to-I editing levels in almost all types of cancers, indicating that this pro-oncogenic ADAR1p150 function helps cancer cells suppress inflammatory responses and thus avoid host immunosurveillance. In contrast to the recent advance in the knowledge of ADAR1p150 functions, the biological functions of the nuclear-localized ADAR1p110, other than its involvement in editing of intronic Alu dsRNAs, have remained mostly unknown.

We have previously described that ADAR1p110 regulates R-loop formation and genome stability at telomeres in cancer cells carrying non-canonical variants of telomeric repeats (FIG. 23). See, WO 2023/004342. ADAR1p110 edits the A-C mismatches within RNA:DNA hybrids formed between canonical and non-canonical variant repeats. Editing of A-C mismatches to I:C matched pairs facilitates resolution of telomeric R-loops by RNase H2. This ADAR1p110-dependent control of telomeric R-loops is required for continued proliferation of telomerase-reactivated cancer cells, revealing the pro-oncogenic nature of ADAR1p110 and identifying ADAR1 as a therapeutic target of telomerase positive cancers.

In certain aspects, methods of treating cancer are provided. In certain embodiments, the cancer cell is selected from those that use telomerase for chromosome maintenance, also referred to herein as a “non-ALT cancer”. Most cancers rely on telomerase to extend and maintain telomeres, but 10-15% of cancers use a homologous recombination-based pathway called alternative lengthening of telomeres (ALT). As used herein, the term “non-ALT cancer” refers to a cancer that relies on telomerase for linear chromosome maintenance and sustained proliferation of telomeres. It is estimated that at least 70-80% of cancer types are non-ALT cancers. Such cancers include, without limitation, melanoma, bladder cancer, Bladder carcinoma, Renal pelvic carcinoma, urothelial carcinoma, Hepatocellular carcinoma, Skin basal cell carcinoma, thyroid cancer (papillary and poorly differentiated carcinomas), myxoid liposarcoma, glioblastoma, medulloblastoma, Oligoastrocytoma, Oligodendroglioma, Breast cancer, colorectal cancer, medullary thyroid carcinoma, ovarian cancer, esophageal adenocarcinoma, acute myeloid leukemia, chronic lymphoid leukemia, pancreatic cancer, prostate cancer, testicular carcinoma, uterine cervix cancer, pleomorphic dermal sarcoma, myxoid liposarcoma, glioma, urothelial cell carcinoma, carcinoma of the skin, liver cancer, gastric cancer, pancreatic cancer, non-small-cell lung cancer and gastrointestinal stromal tumors, prostate cancer, multiple myeloma, recurrent or metastatic breast cancer, solid tumor malignancies, refractory chronic lymphoproliferative disease, non-small-cell lung cancer, HER2⁺ breast cancer, Myelofibrosis, lung, liver, prostate and pancreas, glioblastoma, adenocarcinoma, and acute myelogenous leukemia, See, Jafri et al, Roles of telomeres and telomerase in cancer, and advances in telomerase-targeted therapies, Genome Med. 2016; 8:69. Published online 2016 Jun. 20, which is incorporated herein by reference.

In certain embodiments, the compounds are tested for ADAR1 A-to-I RNA editing using a method described in WO 2023/004342. Briefly, a cancer cell is provided that contains a site for known ADAR1 A-to-I RNA editing, in a highly sensitive and quantitative manner. Adenosine-to-inosine (A-to-I) RNA editing is a co-/posttranscriptional modification of double-stranded RNA (dsRNA), which is catalyzed by the adenosine deaminases acting on RNA (ADAR) family of enzymes and is the most abundant form of RNA editing in higher eukaryotes. ADAR enzymes deaminate adenosine bases to inosine, which is recognized as guanosine by ribosomes and splicing machinery. As such, RNA editing can induce non-synonymous amino acid changes resulting in differential protein isoform expression and thus is considered a key mechanism of transcriptome and proteome diversification in metazoans. For example, ADAR editing of the Q/R site in the GluA2 mRNA modifies a glutamine codon with the consequence that arginine is incorporated since inosine is read as guanosine by the translational machinery. Fritzell et al described the development of a bioluminescent reporter system the highly sensitive and quantitative Nanoluciferase that is conditionally expressed upon reporter-transcript editing of GluA2. Stably introduced into cancer cell lines, the system reports on elevated endogenous ADAR1 editing activity induced by interferon as well as knockdown of ADAR1 and ADAR2 (Fritzell K, Sensitive ADAR editing reporter in cancer cells enables high-throughput screening of small molecule libraries. Nucleic Acids Res. 2019 Feb. 28; 47 (4): e22. doi: 10.1093/nar/gky1228. PMID: 30590609; PMCID: PMC6393238, which is incorporated herein by reference). The dual reporter system includes an adenosine-to-inosine (A-to-I) editing site within a stop codon of a short hairpin dsRNA. The A-to-I editing site is contained within a stop codon (UAG or UGA) at an adenosine base. In the absence of A-to-I editing, transcription is terminated at the stop codon.

The dual reporter is designed such that upon completion of A-to-I editing, the stop codon is recoded to Trp, resulting in in-frame translation of the second reporter, which is downstream of the A-to-I editing site. In the cell line exemplified herein, the reporter utilizes the natural GluA2 editing substrate in which the R/G editing site was modified into a stop codon (UAG) that upon editing is recoded into a tryptophan codon (UGG). A first reporter gene upstream of the edited site monitors translation and a second reporter gene downstream is used to measure read-through after editing. Editing is measured as the ratio between luminescence from the second reporter and the first reporter.

The dual reporter system is designed such that the first reporter is upstream from the adenosine-to-inosine (A-to-I) editing site within a stop codon of a short hairpin dsRNA which is upstream from the second reporter. Each of these components is under control of a single promoter. When A-to-I editing does not occur (in the absence of ADAR1), the stop codon remains in place, and translation stops prior to initiation of translation of the second reporter. When A-to-I editing does occur (in the presence of ADAR1), the stop codon is replaced with a Trp codon, and translation of the second reporter occurs.

### Pharmaceutical Compositions

In another aspect, a pharmaceutical composition comprising a pharmaceutically acceptable carrier and an ADAR1 inhibitor as described herein, is provided. In one embodiment, the ADAR1 inhibitor is selected from

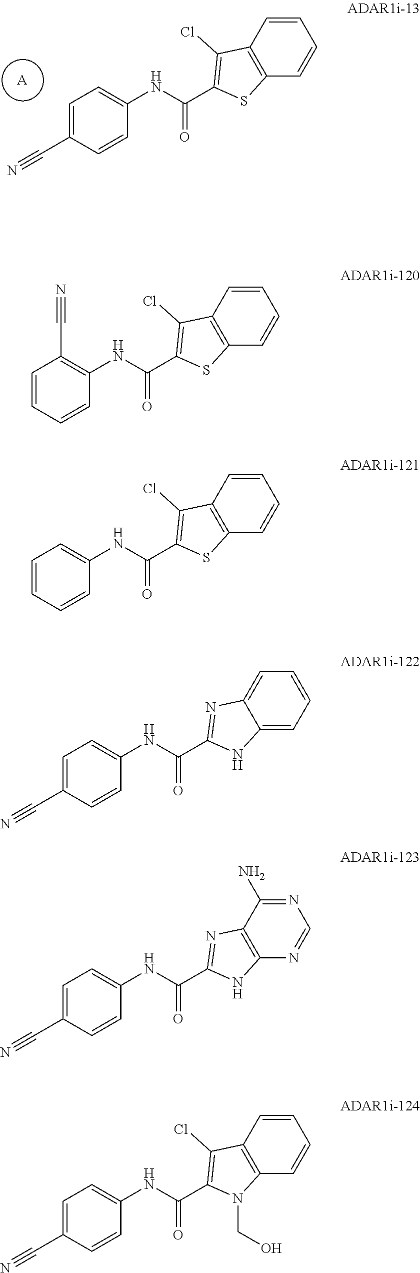

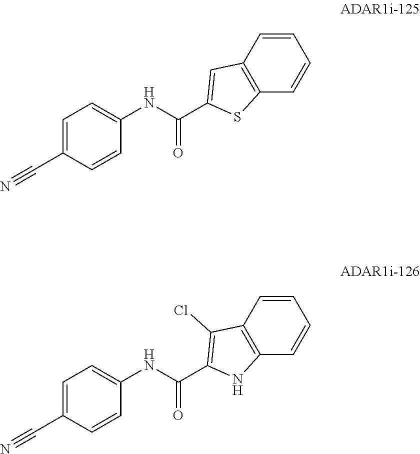 [SMILES: N#Cc1ccc(NC(=O)c2cc3ccccc3s2)cc1 ; N#Cc1ccc(NC(=O)c2[nH]c3ccccc3c2Cl)cc1]

or a prodrug, derivative, pharmaceutical salt, or analog thereof. In another embodiment, the ADAR1 inhibitor is ADARi-13 or a prodrug, derivative, pharmaceutical salt, or analog thereof. In another embodiment, the ADAR1 inhibitor is ADARi-120 or a prodrug, derivative, pharmaceutical salt, or analog thereof. In another embodiment, the ADAR1 inhibitor is ADARi-121 or a prodrug, derivative, pharmaceutical salt, or analog thereof. In another embodiment, the ADAR1 inhibitor is ADARi-122 or a prodrug, derivative, pharmaceutical salt, or analog thereof. In another embodiment, the ADAR1 inhibitor is ADARi-123 or a prodrug, derivative, pharmaceutical salt, or analog thereof. In another embodiment, the ADAR1 inhibitor is ADARi-124 or a prodrug, derivative, pharmaceutical salt, or analog thereof. In another embodiment, the ADAR1 inhibitor is ADARi-125 or a prodrug, derivative, pharmaceutical salt, or analog thereof. In another embodiment, the ADAR1 inhibitor is ADARi-126 or a prodrug, derivative, pharmaceutical salt, or analog thereof.

The terms “analog”, “modification” and “derivative” refer to biologically active derivatives of the reference molecule that retain desired activity as described herein. Preferably, the analog, modification or derivative has at least the same desired activity as the native molecule, although not necessarily at the same level. The terms also encompass purposeful mutations that are made to the reference molecule.

In another embodiment, a combination product is provided that includes a checkpoint inhibitor in addition to the ADAR1 inhibitor. Immune checkpoints represent significant barriers to activation of functional cellular immunity in cancer, and antagonistic antibodies specific for inhibitory ligands on T cells including CTLA4 and programmed death-1 (PD-1) are examples of targeted agents being evaluated in the clinics. In one embodiment, the subject has previously received checkpoint therapy, prior to receiving the ADAR1 inhibitor. The subject may, in some embodiments, receive the same or different checkpoint therapy after administration of the ADAR1 inhibitor.

Immune checkpoint molecules that may be targeted for blocking or inhibition include, but are not limited to, CTLA-4, 4-1BB (CD137), 4-1BBL (CD137L), PDL1, PDL2, PD1, CD134, B7-H3, B7-H4, BTLA, HVEM, TIM3, GAL9, LAG3, TIM3, B7H3, B7H4, VISTA, KIR, 2B4, CD160 (also referred to as BY55) and CGEN-15049. In one embodiment, the checkpoint inhibitor is a PD-1 inhibitor. In another embodiment, the checkpoint inhibitor is a PD-L1 inhibitor.

Immune checkpoint inhibitors include antibodies, or antigen binding fragments thereof, or other binding proteins, that bind to and block or inhibit the activity of one or more of CTLA-4, PDL1, PDL2, PD1, CD134, B7-H3, B7-H4, BTLA, HVEM, TIM3, GAL9, LAG3, TIM3, B7H3, B7H4, VISTA, KIR, 2B4, CD160 and CGEN-15049.

Suitable immune checkpoint inhibitors include those that block PD-1, such as pembrolizumab, nivolumab, AGEN 2034, BGB-A317, BI-754091, CBT-501 (genolimzumab), MEDI0680, MGA012, PDR001, PF-06801591, REGN2810 (SAR439684), and TSR-042. MK-3475 (PD-1 blocker) Nivolumab, and CT-011.

Immune checkpoint inhibitors also include those that block PD-L1, such as durvalumab, atezolizumab, avelumab, and CX-072. Other suitable inhibitors include Anti-B7-H1 (MEDI4736), AMP224, BMS-936559, MPLDL3280A, and MSB0010718C.

Suitable immune checkpoint inhibitors include those that block CTLA-4, such as AGEN 1884, ipilimumab, and tremelimumab.

In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAGS antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-LAG-3 antibody selected from the group consisting of: BMS-986016 and LAG525. In some embodiments, the additional therapeutic agent is an anti-OX-40 antibody selected from: MEDI6469, MEDI0562, and MOXR0916. In some embodiments, the additional therapeutic agent is the anti-4-1BB antibody PF-05082566.

The present disclosure provides compositions and methods that include blockade of immune checkpoints. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAGS), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12 (4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example, it is known that lambrolizumab is also known under the alternative and equivalent names MK-3475 and pembrolizumab.

In addition, more than one immune checkpoint inhibitor (e.g., anti-PD-1 antibody and anti-CTLA-4 antibody) may be used in combination with the ADAR1 inhibitor.

In another embodiment, a combination product is provided which includes an ADAR1 inhibitor in combination with a chemotherapeutic agent, optionally in combination with a checkpoint inhibitor.

Chemotherapeutic agents (e.g., anti-cancer agents) are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol) and docetaxel; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CC→U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; fUdR), and cytarabine (cytosine arabinoside), and purine analogues and related inhibitors such as mercaptopurine (6-mercaptopurine; 6-MP), thioguanine (6-thioguanine; 6-TG), and pentostatin (2′deoxycoformycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,p′-DDD) and aminoglutethimide; analogs thereof derivatives thereof and combinations thereof.

### Methods of Treatment

In one embodiment, the method includes inhibiting or reducing ADAR1 in a subject in need thereof. In one embodiment, the method includes administering an effective amount of an inhibitor of ADAR1. In one embodiment, the ADAR1 inhibitor is ADARi-13 or a prodrug, derivative, pharmaceutical salt, or analog thereof. In another embodiment, the ADAR1 inhibitor is ADARi-120 or a prodrug, derivative, pharmaceutical salt, or analog thereof. In another embodiment, the ADAR1 inhibitor is ADARi-121 or a prodrug, derivative, pharmaceutical salt, or analog thereof. In another embodiment, the ADAR1 inhibitor is ADARi-122 or a prodrug, derivative, pharmaceutical salt, or analog thereof. In another embodiment, the ADAR1 inhibitor is ADARi-123 or a prodrug, derivative, pharmaceutical salt, or analog thereof. In another embodiment, the ADAR1 inhibitor is ADARi-124 or a prodrug, derivative, pharmaceutical salt, or analog thereof. In another embodiment, the ADAR1 inhibitor is ADARi-125 or a prodrug, derivative, pharmaceutical salt, or analog thereof. In another embodiment, the ADAR1 inhibitor is ADARi-126 or a prodrug, derivative, pharmaceutical salt, or analog thereof.

In one embodiment, the effective amount of the ADAR1 inhibitor is an amount ranging from about 0.01 mg/ml to about 10 mg/ml, including all amounts therebetween and end points. In one embodiment, the effective amount of the ADAR1 inhibitor is about 0.1 mg/ml to about 5 mg/ml, including all amounts therebetween and end points. In another embodiment, the effective amount of the ADAR1 inhibitor is about 0.3 mg/ml to about 1.0 mg/ml, including all amounts therebetween and end points. In another embodiment, the effective amount of the ADAR1 inhibitor is about 0.3 mg/ml. In another embodiment, the effective amount of the ADAR1 inhibitor is about 0.4 mg/ml. In another embodiment, the effective amount of the ADAR1 inhibitor is about 0.5 mg/ml. In another embodiment, the effective amount of the ADAR1 inhibitor is about 0.6 mg/ml. In another embodiment, the effective amount of the ADAR1 inhibitor is about 0.7 mg/ml. In another embodiment, the effective amount of the ADAR1 inhibitor is about 0.8 mg/ml. In another embodiment, the effective amount of the ADAR1 inhibitor is about 0.9 mg/ml. In another embodiment, the effective amount of the ADAR1 inhibitor is about 1.0 mg/ml.

In one embodiment, the effective amount of the ADAR1 inhibitor is an amount ranging from about 1 μM to about 2 mM, including all amounts therebetween and end points. In one embodiment, the effective amount of the ADAR1 inhibitor is about 10 μM to about 100 μM, including all amounts therebetween and end points. In another embodiment, the effective amount of the ADAR1 inhibitor is about 5 μM. In another embodiment, the effective amount of the ADAR1 inhibitor is about 10 μM. In another embodiment, the effective amount of the ADAR1 inhibitor is about 20 μM. In another embodiment, the effective amount of the ADAR1 inhibitor is about 50 μM. In another embodiment, the effective amount of the ADAR1 inhibitor is about 100 μM. In another embodiment, the effective amount of the ADAR1 inhibitor is about 200 μM. In another embodiment, the effective amount of the ADAR1 inhibitor is about 300 μM. In another embodiment, the effective amount of the ADAR1 inhibitor is about 400 μM. In another embodiment, the effective amount of the ADAR1 inhibitor is about 500 μM. In another embodiment, the effective amount of the ADAR1 inhibitor is about 600 μM. In another embodiment, the effective amount of the ADAR1 inhibitor is about 700 μM. In another embodiment, the effective amount of the ADAR1 inhibitor is about 800 μM. In another embodiment, the effective amount of the ADAR1 inhibitor is about 900 μM. In another embodiment, the effective amount of the ADAR1 inhibitor is about 1 mM. In another embodiment, the effective amount of the ADAR1 inhibitor is about 1.25 mM. In another embodiment, the effective amount of the ADAR1 inhibitor is about 1.5 mM. In another embodiment, the effective amount of the ADAR1 inhibitor is about 1.75 mM. In another embodiment, the effective amount of the ADAR1 inhibitor is about 2 mM.

In certain embodiments, the subject is also treated with an effective amount of the checkpoint inhibitor. The checkpoint inhibitor can be any of those known in the art, or described herein. It should be understood that the “effective amount” for the checkpoint inhibitor may vary depending upon the agent(s) selected for use in the method, and may be determined by the person of skill in the art. In one embodiment an effective amount for the checkpoint inhibitor includes without limitation about 1 μg to about 25 mg. In one embodiment, the range of effective amount is 0.001 to 0.01 mg. In another embodiment, the range of effective amount is 0.001 to 0.1 mg. In another embodiment, the range of effective amount is 0.001 to 1 mg. In another embodiment, the range of effective amount is 0.001 to 10 mg. In another embodiment, the range of effective amount is 0.001 to 20 mg. In another embodiment, the range of effective amount is 0.01 to 25 mg. In another embodiment, the range of effective amount is 0.01 to 0.1 mg. In another embodiment, the range of effective amount is 0.01 to 1 mg. In another embodiment, the range of effective amount is 0.01 to 10 mg. In another embodiment, the range of effective amount is 0.01 to 20 mg. In another embodiment, the range of effective amount is 0.1 to 25 mg. In another embodiment, the range of effective amount is 0.1 to 1 mg. In another embodiment, the range of effective amount is 0.1 to 10 mg. In another embodiment, the range of effective amount is 0.1 to 20 mg. In another embodiment, the range of effective amount is 1 to 25 mg. In another embodiment, the range of effective amount is 1 to 5 mg. In another embodiment, the range of effective amount is 1 to 10 mg. In another embodiment, the range of effective amount is 1 to 20 mg. Still other doses falling within these ranges are expected to be useful. The effective amount of the checkpoint inhibitor may be individually chosen based on the agent selected and other factors, e.g., size of the patient, type of cancer, etc.

In one embodiment, the ADAR1 inhibitor and checkpoint inhibitor are administered approximately simultaneously. In another embodiment, the ADAR1 inhibitor are administered prior to checkpoint inhibitor. In another embodiment, the ADAR1 inhibitor are administered subsequent to the checkpoint inhibitor.

In another embodiment, the method includes administering a chemotherapeutic agent to the subject in addition to the ADAR1 inhibitor, and optionally with a checkpoint inhibitor. Effective dosages for individual chemotherapeutic agents can be determined by the person of skill in the art.

In certain embodiments, the cancer being treated is a non-ALT cancer. In certain embodiments, the cancer treated includes, but is not limited to, a solid tumor, a hematological cancer (e.g., leukemia, lymphoma, myeloma, e.g., multiple myeloma), and a metastatic lesion. In one embodiment, the cancer is a solid tumor. Non-limiting examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, non-small cell lung cancer, cancer of the small intestine, cancer of the esophagus. The cancer may be at an early, intermediate, late stage or metastatic cancer.

The chemotherapeutic agents, ADAR1 inhibitors and checkpoint inhibitors may be administered using any suitable route of administration. For example, compositions may be administered via intravenous, parenteral, subcutaneous, intramuscular, intracranial, intraorbital, ophthalmic, intraventricular, intracapsular, intraspinal, intracisteral, intraperitoneal, intranasal, or aerosol administration. The route of administration for each composition (e.g., ADAR1 inhibitors, chemotherapeutic agents, checkpoint inhibitors) may be determined individually and may be the same or different.

A “subject” is a mammal, e.g., a human, mouse, rat, guinea pig, dog, cat, horse, cow, pig, or non-human primate, such as a monkey, chimpanzee, baboon or gorilla. The term “patient” may be used interchangeably with the term subject. In one embodiment, the subject is a human. The subject may be of any age, as determined by the health care provider. In certain embodiments described herein, the patient is a subject who has previously been diagnosed with cancer. The subject may have been treated for cancer previously, or is currently being treated for cancer. In one embodiment, the subject has a non-ALT cancer, e.g., a telomerase-activated cancer.

As used herein, the term “a therapeutically effective amount” refers an amount sufficient to achieve the intended purpose. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

In yet another embodiment, the methods described herein include treatment in combination with another cancer treatment or therapeutic agent to reduce or inhibit reversal of cancer cell dormancy, including known chemotherapeutic agents. The reduction or inhibition of cancer cell proliferation can be measured relative to the incidence observed in the absence of the treatment. The tumor inhibition can be quantified using any convenient method of measurement. Tumor inhibition can be reduced by about 10%, 20%, 30%, 40%, 50%, 60%, 70%, 80%, 90%, 95% or greater.

It is to be noted that the term “a” or “an” refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### EXAMPLES

### Example 1: Materials and Methods

### Preparation of Duplex Substrates

Sense or antisense oligonucleotides of telomere sequences were purchased from IDT and Dharmacon. The 5′ ends of RNA and DNA strands to be analyzed were biotinylated. Sense and antisense oligonucleotides were annealed in annealing buffer (10 mM Tris-HCl pH 7.5, 50 mM NaCl) to prepare perfectly matched or mismatched dsRNAs or RNA:DNA hybrids, which were used as substrates for in vitro editing assay.

### Preparation of Recombinant ADAR1 Proteins

All procedures were carried out at 4° C. HAT-ADAR1p110-WT-, FLAG-ADAR1p110-WT-, or HA-ADAR1p110-EAA-expressing Sf9 cells were prepared with baculovirus⁶⁹. The cells were washed with PBS and resuspended in Tris+buffer (250 mM Tris pH 7.8, 1 mM dithiothreitol (DTT), 0.6 mM phenylmethylsulfonyl fluoride (PMSF), proteinase inhibitor cocktail). The cells were sonicated and debris was removed by centrifugation. The supernatant (cell extract) was diluted with an equal volume of 2×TGK buffer (100 mM Tris-HCl pH 7.8, 200 mM NaCl, 40% glycerol, 1 mM DTT, 0.6 mM PMSF, proteinase inhibitor cocktail) and stored at −80° C.

HAT-ADAR1p110-WT was purified using TALON Metal Affinity Resin (Clontech). The resin was prewashed with STD300 buffer (50 mM Tris-HCl pH 7.0, 300 mM NaCl, 20% glycerol, 1 mM β-mercaptoethanol, 0.05% NP-40). After buffer exchange to STD300 using Zeba™ 7 K molecular weight cut-off (MWCO) spin desalting column (Thermo Fisher), the cell extract was loaded onto the resin. After washing with STD300 buffer, the resin was treated with 80 kU of micrococcal nuclease (NEB) for 30 min in STD300 buffer containing 1 mM CaCl₂) at room temperature and washed with STD300 buffer containing 0.5 mM EDTA and 0.5 mM EGTA and then washed with STD300 buffer containing 7.5 mM imidazole. HAT-ADAR1p110-WT recombinant protein was eluted with STD300 buffer containing 150 mM imidazole and proteinase inhibitor cocktail. Imidazole was removed by using Zeba™ 7 K MWCO spin desalting column.

FLAG-ADAR1p110-WT or HA-ADAR1p110-EAA was purified using anti-FLAG M2 agarose (Sigma) or anti-HA agarose (Thermo Fisher Scientific), respectively. The agarose was washed with STD150 buffer (50 mM Tris-HCl pH 7.0, 150 mM NaCl, 20% glycerol, 1 mM β-mercaptoethanol, 0.05% NP-40). After buffer exchange to STD150 using Zeba™ 7 K MWCO spin desalting column, the cell extract was loaded onto the agarose. After washing with STD150 buffer, the agarose was treated with 80 kU of micrococcal nuclease (NEB) for 30 min in STD150 buffer containing 2 mM CaCl₂) at room temperature and washed with STD150 buffer containing 3 mM EDTA and 3 mM EGTA, STD150 buffer, STD500 buffer (50 mM Tris-HCl pH 7.0, 500 mM NaCl, 20% glycerol, 1 mM β-mercaptoethanol, 0.05% NP-40), and again STD150 buffer. FLAG-ADAR1p110-WT or HA-ADAR1p110-EAA recombinant protein was eluted with STD150 buffer containing protease inhibitor cocktail and 0.1 mg/ml FLAG peptide or HA peptide, respectively.

All recombinant proteins purified were stored in STD150 buffer containing 1 mM DTT, instead of 1 mM β-mercaptoethanol, at −80° C.

### In Vitro Editing Assay

The in vitro editing reaction mixture, containing 5 nM of telomere RNA:RNA duplex substrates and 75 nM of HAT-ADAR1p110-WT, FLAG-ADAR1p110-WT, or HA-ADAR1p110-EAA protein, was incubated at 37° C. for 2 h in in vitro editing buffer I (20 mM HEPES-KOH PH 7.5, 100 mM NaCl, 0.01% NP-40, 5% glycerol, 1 mM DTT). For editing of RNA:DNA hybrid substrates, in vitro editing buffer II (20 mM HEPES-KOH PH 7.5, 20 mM NaCl, 0.01% NP-40, 5% glycerol, 1 mM DTT) was used. Edited RNA or DNA strands were purified using Dynabeads MyOne Streptavidin C1 (Thermo Fisher Scientific). To remove opposite RNA or DNA strands, rNase H (NEB) or TURBO dNase (Thermo Fisher Scientific) was used, respectively. For sequencing of edited substrates, reverse transcription-PCR was carried out for RNA strands, while PCR was carried out for DNA strands. RT reactions were carried out using SuperScript III Reverse Transcriptase (Thermo Fisher Scientific), and PCR reactions were performed using Platinum Taq DNA polymerase (Thermo Fisher Scientific). PCR products were sequenced using a specific sequencing primer, and the ratio of A and G peaks in the chromatograms were analyzed by CodonCode Aligner (CodonCode Corporation).

Preparation of rNase H2 Complex and RNA:DNA Hybrid Cleavage Assay

To prepare recombinant human rNase H2A/2B/2C triple complex, pET28-FLAG-rNaseH2A and pET15-His-rNaseH2B/2C vectors were co-transformed into E. coli BL21 cells cultured in LB medium containing 100 μg/ml ampicillin and 20 μg/ml kanamycin. Protein induction was started at an optical density of 0.6 with 0.4 mM isopropyl β-D-1-thiogalactopyranoside and incubated overnight at 30° C. After harvesting cells by centrifugation, cells were suspended in 40 mM HEPES pH 7.0, 75 mM NaCl, 5% glycerol, proteinase inhibitor cocktail, and 1 mg/ml lysozyme and then sonicated. The debris was removed by centrifugation twice at 12,000×g. The supernatant was diluted with an equal volume of dilution buffer (40 mM NaH₂PO₄ pH 7.0, 500 mM NaCl, 5% glycerol) and mixed with Ni-NTA agarose (Qiagen) at 4° C. The agarose was washed with STD300 buffer containing 5 mM imidazole. Recombinant rNase H2A/2B/2C complex was eluted with 20 ml of STD300 buffer containing 150 mM imidazole and proteinase inhibitor cocktail. Anti-FLAG M2 affinity gel (Sigma) was washed with STD150 buffer and mixed with an eluted fraction of Ni-NTA agarose at 4° C. After washing with STD150 buffer, the resin was treated with 80 kU of micrococcal nuclease (NEB) for 30 min in STD150 buffer containing 2 mM CaCl₂) at room temperature and washed with STD500 buffer and then with STD150 buffer. FLAG-rNase H2A/His-rNase H2B/2C complexes were eluted with 0.1 mg/ml FLAG peptide in STD150 buffer containing proteinase inhibitor cocktail.

5′-³²P-labeled oligonucleotide RNAs were annealed with complementary DNAs as described above. Cleavage assays of RNA:DNA hybrid by rNase H2A/2B/2C complex were done in a 50 μl reaction mixture containing 50 mM Tris-HCl pH 8.5, 75 mM KCl, 3 mM MgCl₂, 10 mM DTT, 2 nM rNase H2A/2B/2C complex, 1 nM RNA:DNA substrate, and rNasin plus inhibitor. Cleavage assays by recombinant human rNase H1 (ab153634, Abcam) were done in a 50 μl reaction mixture containing 25 mM Tris-HCl pH 7.5, 50 mM KCl, 5 mM MgCl₂, 1 mM DTT, 10 μg/ml BSA, 5 nM rNase H1, 1 nM RNA:DNA substrate, and rNasin plus inhibitor. Reaction mixtures were incubated at 37° C. and 7.5 μl aliquots were taken after 0, 5, 10, 30, and 60 min. At each time point, to stop the reaction, gel loading buffer (80% formamide, 20% glycerol, 0.025% bromophenol) was added to the aliquots. After heating at 80° C. for 10 min, samples were analyzed by 10% Urea-PAGE. 5′-32P-labeled RNA signals were detected using a Typhoon RGB Imager (GE Healthcare, Amersham Typhoon Control software).

### Statistics and Reproducibility

All experiments were performed at least twice or more independent times with similar results. Image quantitation was done using Image J or ImageQuant software (GE Healthcare). Data were analyzed using Microsoft Excel (Microsoft Corporation) and were presented as means±SD or SEM. Two-tailed t tests were conducted where the minimum level of significance was P<0.05.

### Example 2: Multi-Step Screening Strategy—First Screen

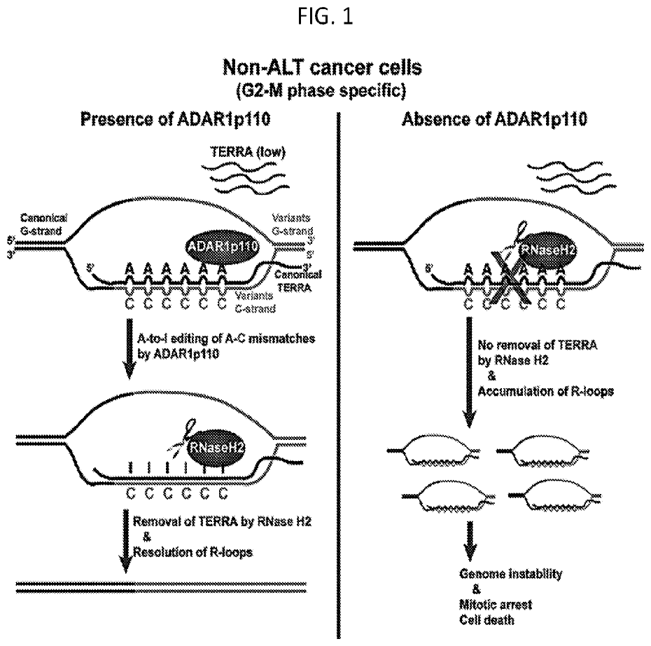

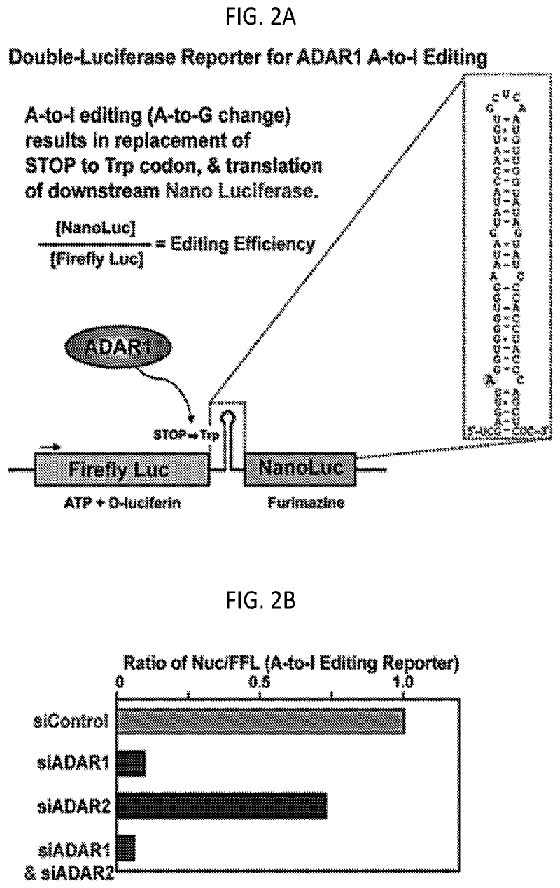

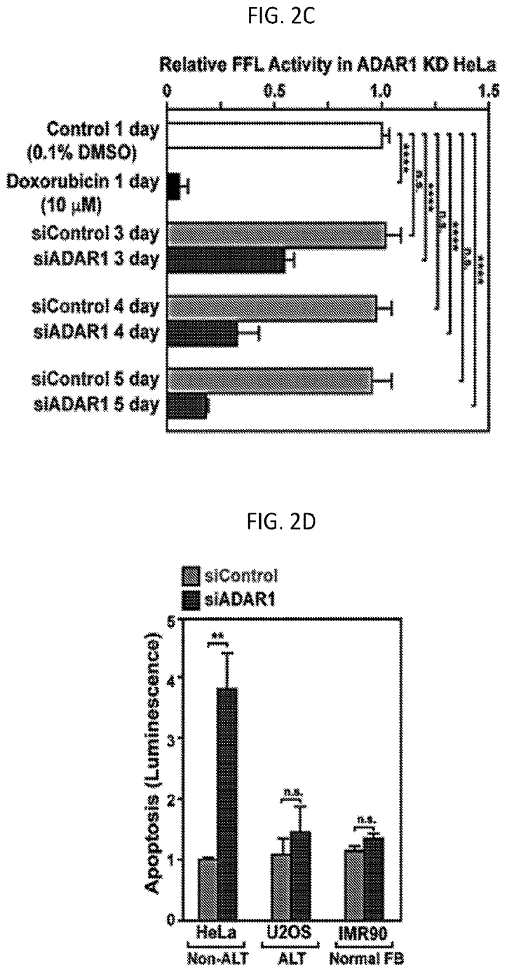

Our results indicate that inhibition of the ADAR1 A-to-I editing activity results in accumulation of telomeric R-loops specifically in telomerase reactivated cancer cells and consequently their apoptotic cell death. (FIG. 1) Using a permanently transformed HeLa cell line, we confirmed that this reporter system (FIG. 2A) can be used to monitor very specifically the ADAR1-mediated A-to-I RNA editing activity (FIG. 2B); the reporter activity was reduced only in ADAR1 depleted cells (FIG. 2B). We noted that inhibition of ADAR1 editing activity resulted in “slow” induction of apoptosis due to telomere instability and mitotic arrest, which can be measured as reduction in expression of Firefly Luciferase (FFL) (FIG. 2C), specifically in non-ALT cancer cells (FIG. 2D).

A HeLa cell line (telomerase positive) permanently transformed with a dual luciferase reporter system (HeLa-Nluc-edit) as described by Fritzell, K. et al (Sensitive ADAR editing reporter in cancer cells enables high-throughput screening of small molecule libraries. Nucleic acids research 47, e22, doi: 10.1093/nar/gky1228 (2019), which is incorporated herein by reference) were seeded at a density of 800 cells per well into 384 white plates (PerkinElmer, CulturPlate384, #6007689) in 20 μl of Opti-MEM without phenol red (Thermo Fisher Scientific #11058021) using a multidrop dispenser (Thermo Fisher Scientific) and incubated overnight at 37° C., 5% CO2. The cells were then treated with test compounds (Maybridge HitFinder Diversity Set library) for 72 h prior to luminescence signal detection. Briefly, 10 μl of Steadylite Plus FFL assay system reagents were added and firefly luminescence signal was measured within 10-20 min after the addition using a multiplate reader at 1 s integration time. Firefly luciferase activity was calculated for each well and results for each plate were normalized to a negative (0.1% DMSO) and positive (Doxorubicin 10 microM) control.

Approximately 1,600 compounds were identified from 14,400, which showed at least a 30% decrease (at least 30% and up to 100% decrease) of FFL activity as compared to the control, which was used as a measure of cell viability/apoptosis. These compounds were further tested in the subsequent screens.

### Example 3: Multi-Step Screening Strategy—Second and Third Screens

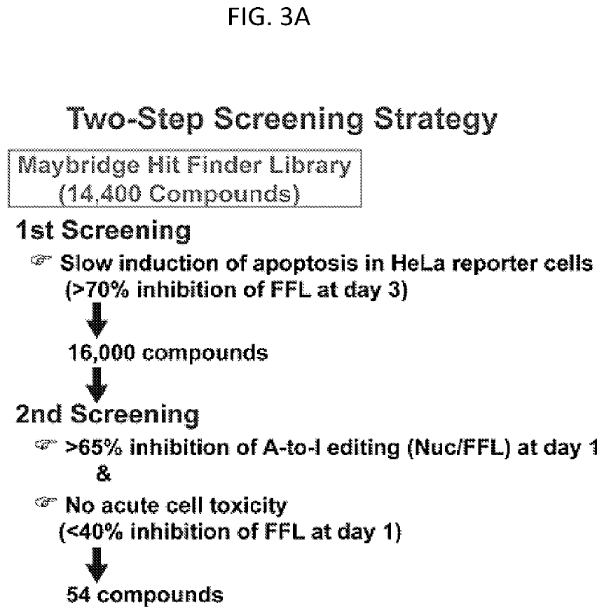

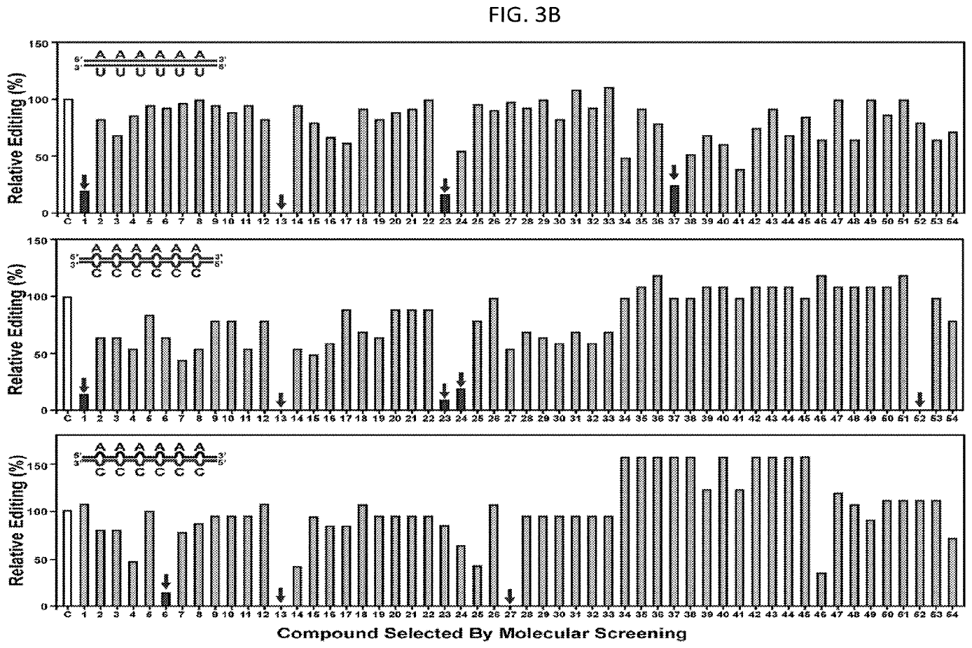

A schematic of the final two-step screening strategy using the dual luciferase reporter system (2nd and 3rd screens) is shown in FIG. 3A. Inhibition of A-to-I editing activity measured as the reduction of Nano Luciferase activity (ratio of Nuc vs FFL) at day 1 was used as the second screening. The absence of extreme and acute cytotoxicity (<40% reduction of FFL at day 1) was also taken into account. The second step screening resulted in identification of 54 candidate compounds. (FIG. 3B) Using our well-established in vitro A-to-I editing assay, 54 preselected compounds were evaluated for their ADAR1 inhibitory effects using recombinant ADAR1p110 and synthetic dsRNA substrates, known to be targeted by both ADAR1 and ADAR2, as described previously. Although ADAR1 does edit A:U base pairs of completely matched dsRNAs, A-C mismatched base pairs are in fact the favored ADAR1 target sites. Therefore, both matched (top) and mismatched (bottom) dsRNA substrates were tested. Note that some compounds (compound #13) preferentially inhibited matched adenosines, whereas the others (compound #52) had more specific inhibitory effects on adenosine at A-C mismatched base pairs. We are currently testing their inhibitory effects on RNA:DNA hybrid substrates.

HeLa-Nluc-edit cells were seeded at a density of 15,000 cells per well into 384 white plates (PerkinElmer, CulturPlate384, #6007689) in 20 μl of Opti-MEM without phenol red (Thermo Fisher Scientific #11058021) using a multidrop dispenser (Thermo Fisher Scientific) and incubated overnight at 37° C., 5% CO2. The cells were then treated with the 1,600 test compounds identified in the First Screen (Example 3) for 24 h prior to luminescence signal detection.

For the second screen, 10 μl of Steadylite Plus FFL assay system reagents were added and firefly luminescence signal was measured within 10-20 min after the addition using a multiplate reader at 1 s integration time. Firefly luciferase activity was calculated for each well and results for each plate were normalized to a negative (0.1% DMSO) and positive (Doxorubicin 10 microM) control.

For the third screen, 20 μl of ONE-Glo EX reagent (Nano Glo Dual Luciferase assay) was added and luminescence signal was measured within 10-20 min after the addition using a multiplate reader at 1 s integration time. Subsequently, 20 μl NanoDLR Stop & Glo Reagent (Nano Glo Dual Luciferase assay) (Promega) were added to the same wells and luminescence signal was measured with Envision (PerkinElmer) multiplate reader at 0.1 s integration time, at least 10 min after reagent addition. The ratio between Nluc and FFL signal was calculated for each well and results for each plate were normalized to a negative (0.1% DMSO) and positive (Nluc inhibitor 1 10 microM) control.

54 compounds were selected from the 1,600 compounds identified in the first screen (FIG. 3B). These compounds showed less than 40% decrease of FFL activity (2^{nd} selection) & more than 60% inhibition of the reporter activity (Nluc/FFL=A-to-I editing activity) (3^{rd} selection).

### Example 4: In Vitro Screen

In a previous study, the 54 compounds identified in the high-throughput screen were further tested in the in vitro editing assay described in Examples 2 and 3. The in vitro editing reaction mixture, containing 5 nM of telomere RNA:RNA duplex substrates and 75 nM of HAT-ADAR1p110-WT, FLAG-ADAR1p110-WT, or HA-ADAR1p110-EAA protein, was incubated at 37° C. for 2 h in in vitro editing buffer I (20 mM HEPES-KOH PH 7.5, 100 mM NaCl, 0.01% NP-40, 5% glycerol, 1 mM DTT) with 10 microM test compound. For editing of RNA:DNA hybrid substrates, in vitro editing buffer II (20 mM HEPES-KOH PH 7.5, 20 mM NaCl, 0.01% NP-40, 5% glycerol, 1 mM DTT) was used. Edited RNA or DNA strands were purified using Dynabeads MyOne Streptavidin C1 (Thermo Fisher Scientific). To remove opposite RNA or DNA strands, Rnase H (NEB) or TuRBO Dnase (Thermo Fisher Scientific) was used, respectively. For sequencing of edited substrates, reverse transcription-PCR was carried out for RNA strands, while PCR was carried out for DNA strands. RT reactions were carried out using SuperScript III

Reverse Transcriptase (Thermo Fisher Scientific), and PCR reactions were performed using Platinum Taq DNA polymerase (Thermo Fisher Scientific). PCR products were sequenced using a specific sequencing primer, and the ratio of A and G peaks in the chromatograms were analyzed by CodonCode Aligner (CodonCode Corporation).

### Example 5: Identification of Additional ADAR1 Inhibitor Compounds

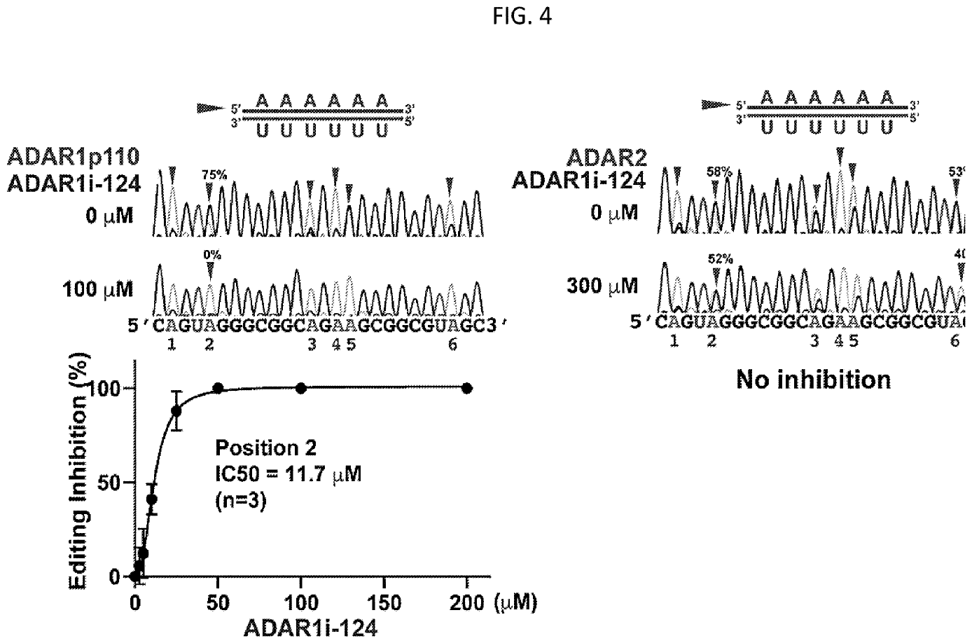

Eight of the “hit” compounds were tested for in vitro editing inhibition dose dependency, selective cancer cell killing, and their potency for induction of DNA damage, M phase arrest, and apoptosis. Compound #13 (3-chloro-N-(4-cyanophenyl)-1-benzothiophene-2-carboxamide, renamed ADARi-13) was selected for further studies, as only ADAR1i-13 inhibited ADAR1p110 editing of all three substrates tested, including the DNA:RNA hybrid substrate. FIG. 4

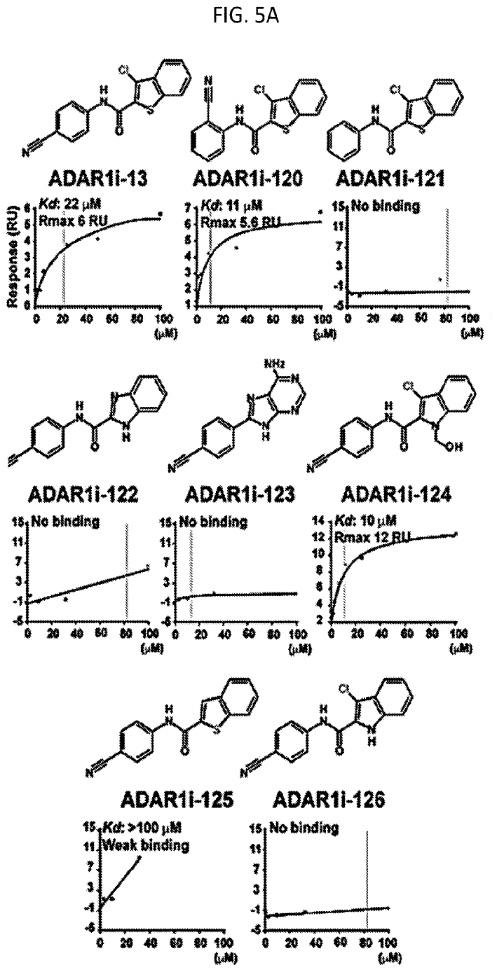

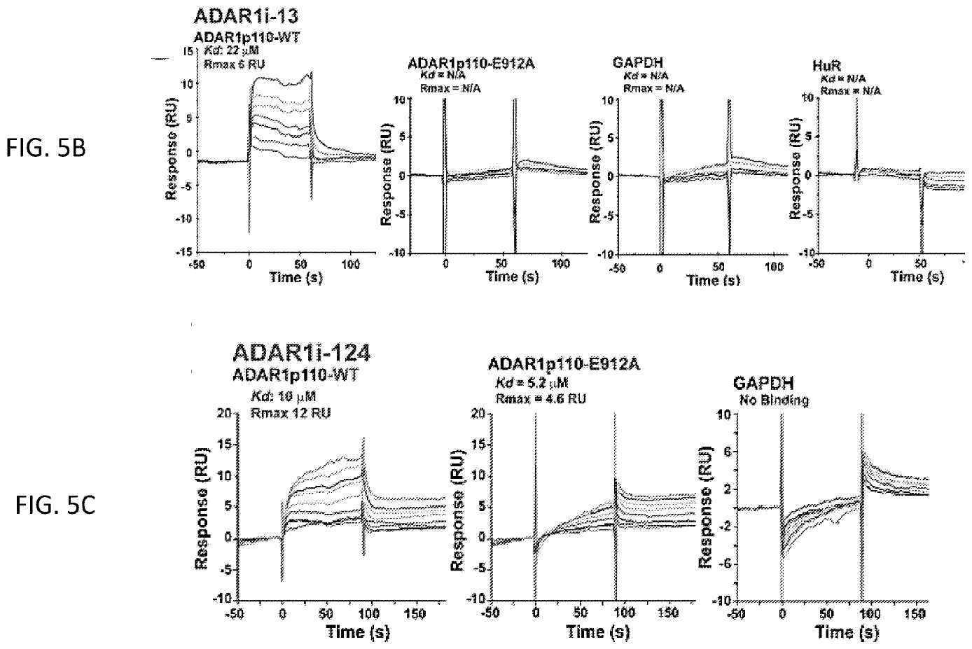

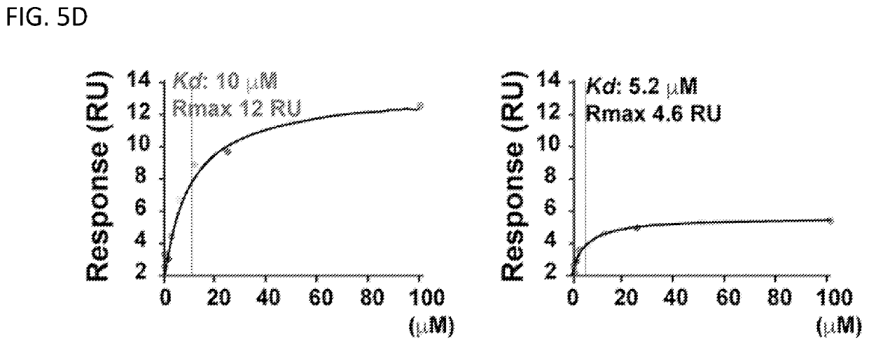

Seven additional analogs of ADAR1i-13 (FIG. 5A) were synthesized to determine structural characteristics required for ADAR1 editing inhibitory activity. In vitro editing assay revealed ADAR1 inhibitory activities of two analogs ADAR1i-120 and ADAR1i-124. In vitro editing IC50 values of ADAR1i-13, ADAR1i-120, and ADAR1i-124 (FIG. 5A) were similar, in the ~10-20 μM range, for both ADAR1p110 (FIG. 5A) and ADAR1p150 (not shown). No ADAR2 inhibitory activity was detected with these 3 compounds, as an example assay shown for ADAR1i-124 (FIG. 4). Examination of editing inhibition positive and negative analog compounds revealed the importance of the cyano and chloride substituents for A-to-I editing inhibition (FIG. 5A).

### Example 6: Direct Binding of ADAR1i-13 and ADAR1i-124 to the Catalytic Center of ADAR1p110

No complete 3-D structure of ADAR1 is currently available. However, structural features critical for the ADAR deamination reaction have been revealed. The catalytic center of ADAR contains a zinc ion coordinated by one histidine and two cysteine residues, revealed by site directed mutagenesis of ADAR1 (42) and the X-ray crystallography studies on ADAR2 catalytic domain (43). The glutamate E912 and E396 in ADAR1 and ADAR2, respectively, plays an important proton transfer function during hydrolytic deamination reaction (42, 43). Surface plasmon resonance (SPR) analysis confirmed direct binding of 3 editing inhibitors, ADAR1i-13, ADAR1i-120, and ADAR1i-124, to ADAR1p110-WT (FIG. 5A). Interestingly, ADAR1i-13 did not bind to the ADAR1p110-E912A mutant (FIG. 5B), whereas ADAR1i-124 showed a binding kinetics different from that with ADAR1p110-WT (FIG. 5C). These results indicate that both inhibitors interact with E912 in the catalytic center pocket.

Importantly, ADAR1i-13 and ADAR1i-124 did not bind to negative control proteins GAPDH and HuR (FIGS. 5B, 5C) and ADAR1i-122, -123, -125, and -126 carrying the cyano group did not bind to ADAR1p110-WT (FIG. 5A), indicating that the cyano group, despite of its potential to make a covalent bonding, is not causing non-specific binding (FIG. 5A). Notably, both ADAR1i-13 and ADAR1i-124 bound also to ADAR2 (not shown), although they did not inhibit ADAR2 mediated A-to-I editing in vitro (FIG. 4). These results perhaps indicate the importance of the precise way of fitting of the compound into the catalytic center pocket.

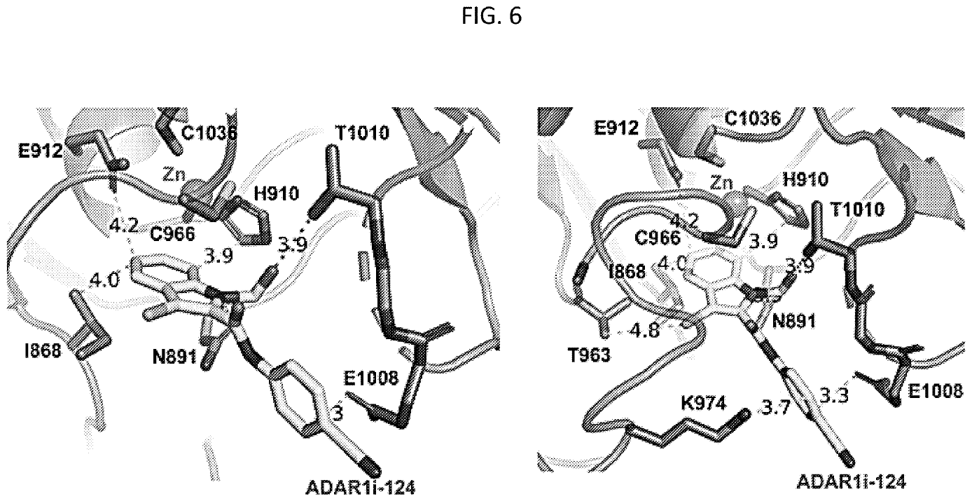

Computer simulation of ADAR1i-124 docking revealed that the compound indeed fits well into the active site, near the zinc atom, and makes close contacts to several amino acid residues including E912, T963 interacting with the chloride and T1010 interacting with the hydroxyl methyl group (FIG. 6). The cyano group extends out from the catalytic center pocket, possibly interfering with access of dsRNA into the active site.

### Example 7: In Vivo Inhibition of ADAR1-Mediated Editing Activity Results in Oligomerization of MDA5-dsRNA Complexes and Formation of Fiber Structures

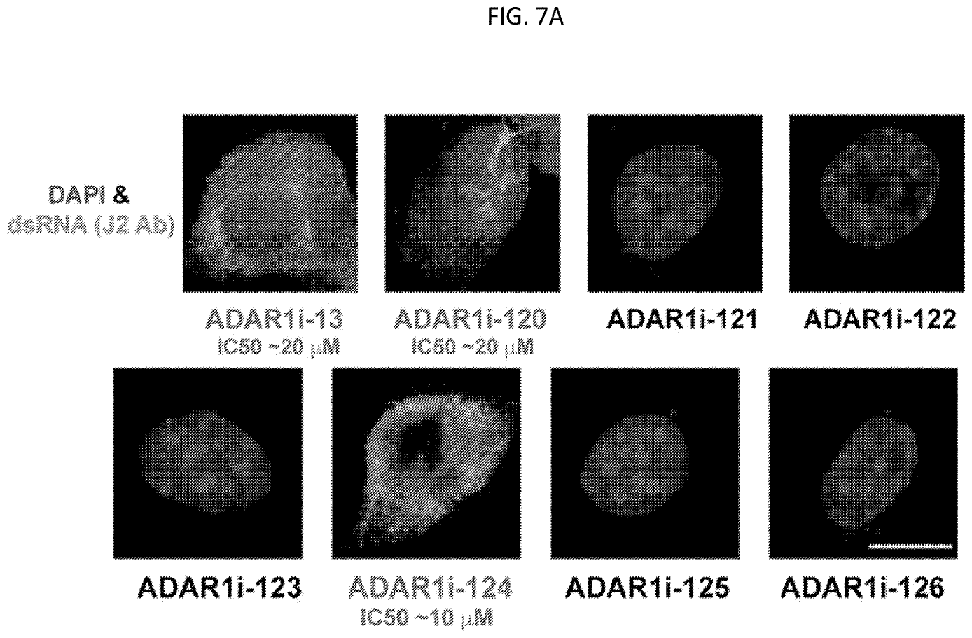

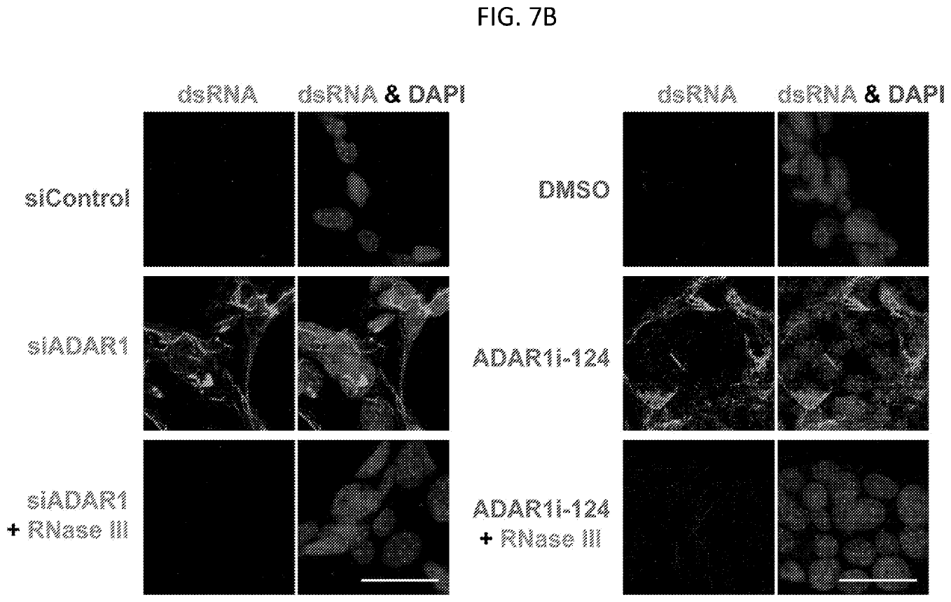

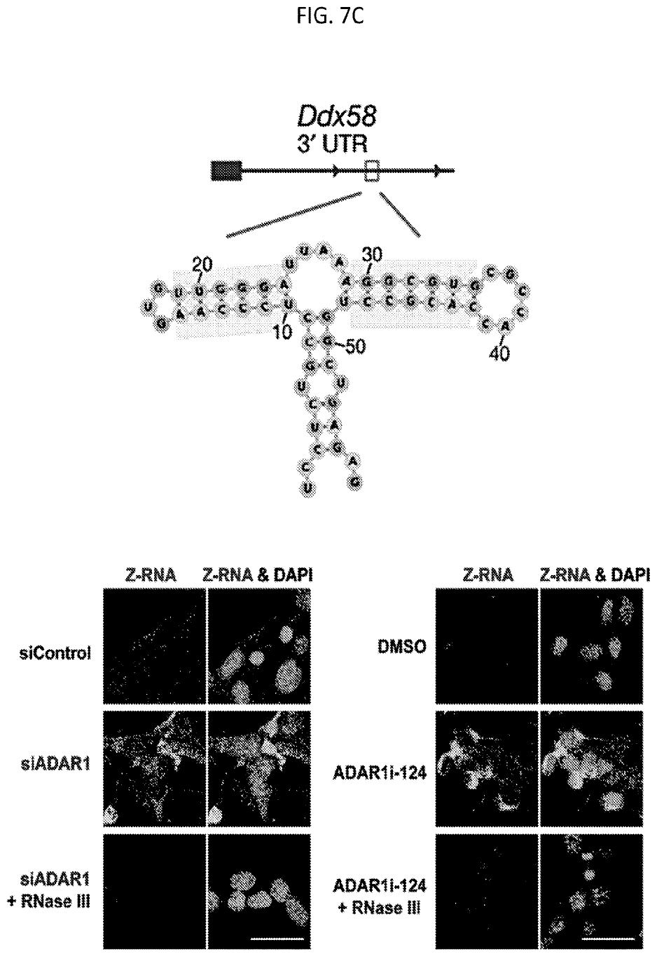

It has been reported that the suppression ADAR1 editing activity results in binding of MDA5 to unedited dsRNAs, oligomerization of MDA5-dsRNA complexes, and formation of fiber-like structures (21, 44). These dsRNA fiber structures were detected in cells treated with ADAR1 inhibition active compounds, ADAR1i-13, ADAR1i-120, and ADAR1i-124, but not detected in cells treated with ADAR1 inhibition negative compounds (FIG. 7A). The dsRNA fibers induced by ADAR1 knockdown and also by ADAR1i-124 were abolished by RNase III treatment (FIG. 7B). The results demonstrate in vivo inhibition of ADAR1 editing activity by three compounds with their editing inhibitory activity already confirmed in vitro.

### Example 8: Selective Killing of Cancer Cells and Induction of IFN Signaling by ADAR1i-124

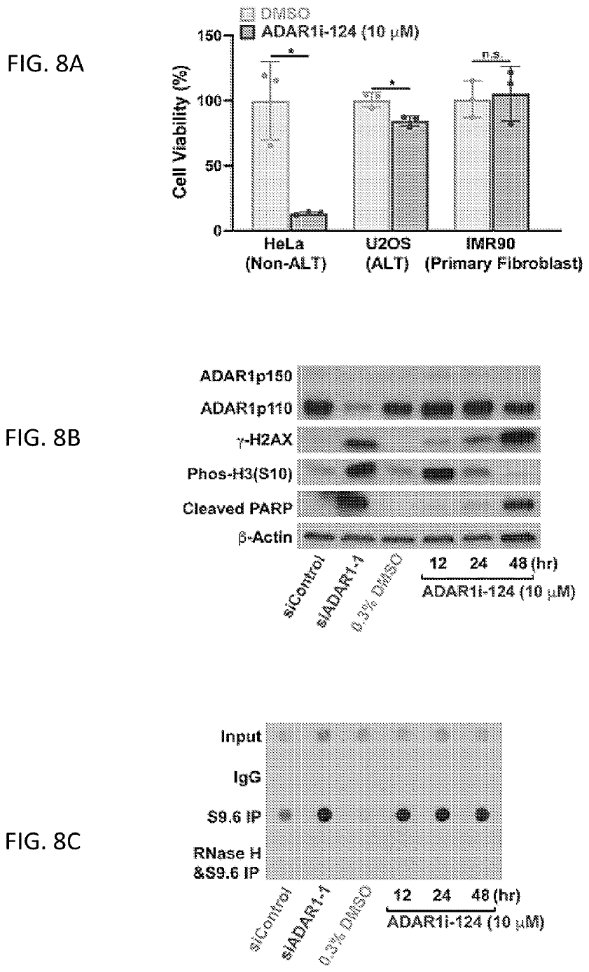

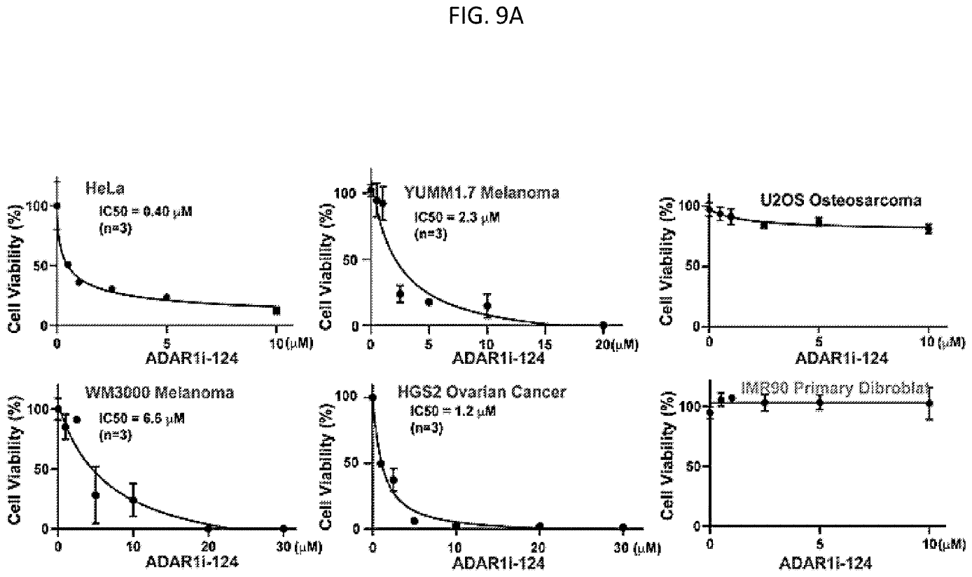

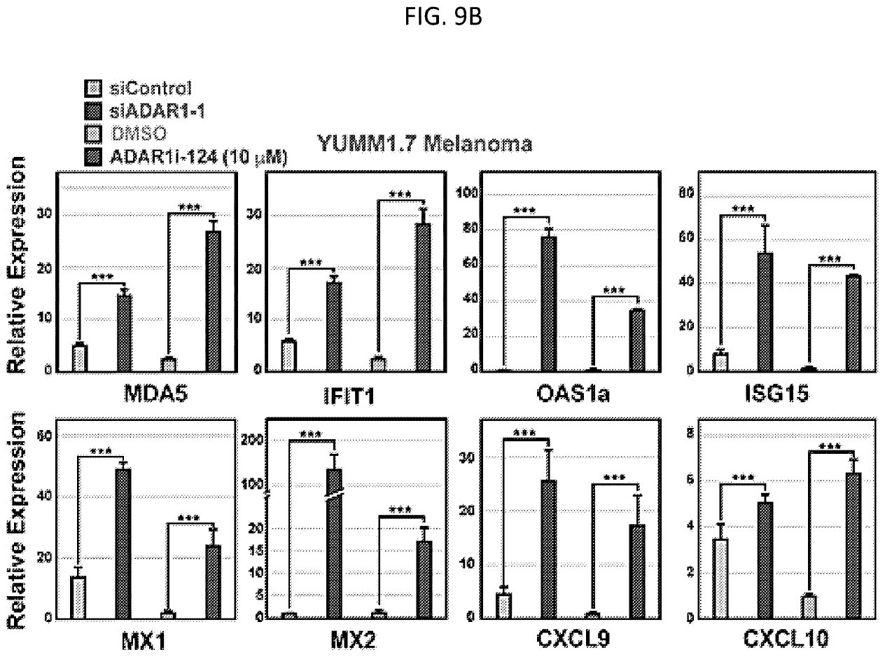

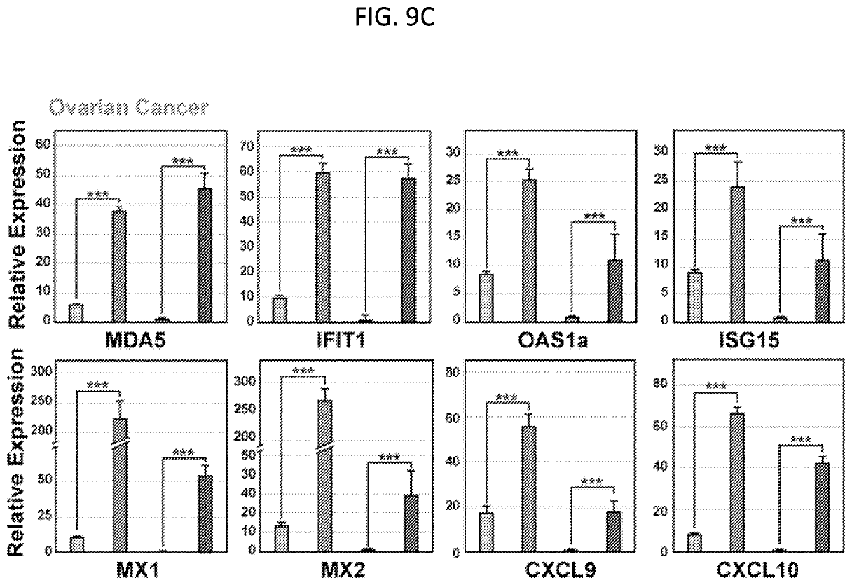

We previously found that acute knockdown of ADAR1 resulted in accumulation of R-loops and extensive DNA damage at telomeres and consequently M phase arrest and apoptosis of non-ALT (or telomerase reactivated) cancer cells (3). All three ADAR1 inhibitor compounds ADAR1i-13, ADAR1i-120, and ADAR1i-124 indeed killed selectively non-ALT HeLa cells but not ALT U2OS cells or IMR90 normal fibroblast cells (FIG. 8A) and induced DNA damage, M phase arrest, and apoptosis (FIG. 8B), and increased formation of R-loops (RNA:DNA hybrids) (FIG. 8C), as shown for ADAR1i-124. Dose-dependent selective killing of telomerase reactivated cancer cells was determined (FIG. 9A). ADAR1i-124 suppressed human and mouse melanoma (telomerase reactivated) cell lines more efficiently than ADAR1i-13 and ADAR1i-120 (not shown), i.e., ADAR1i-124 had the lowest IC50 (~0.4 μM) for selective killing of HeLa cells (FIG. 9A). Finally, ADAR1i-124, like siADAR1, upregulated expression of interferon stimulated genes (ISGs) including MDA5, CXCL9, and CXCL10 in YUMM1.7 cells (FIG. 9B), indicating that ADAR1i-124 induced the IFN signaling pathway, prerequisite for effective ICB (2).

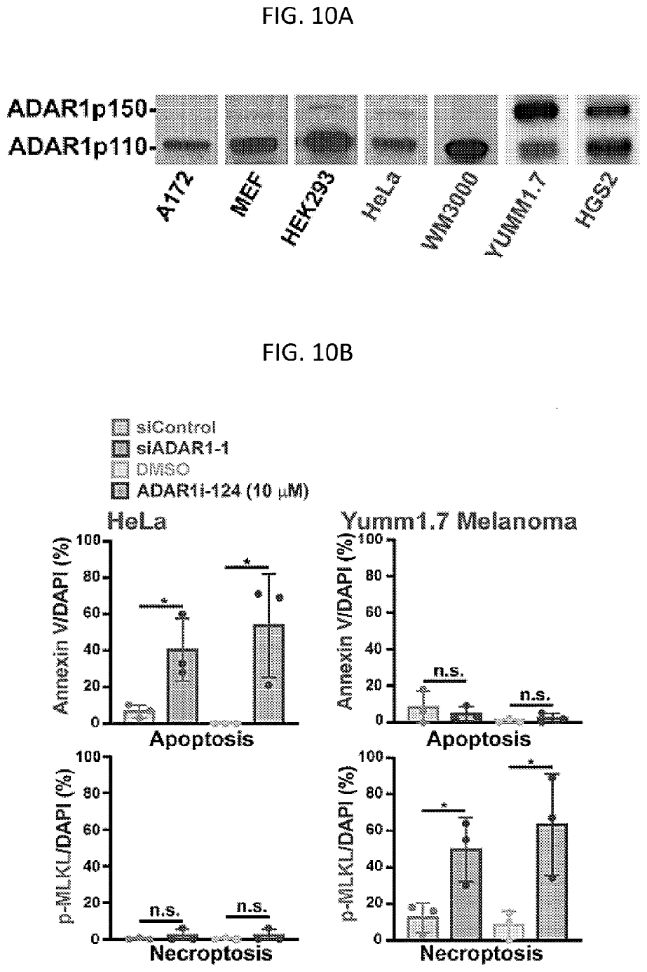

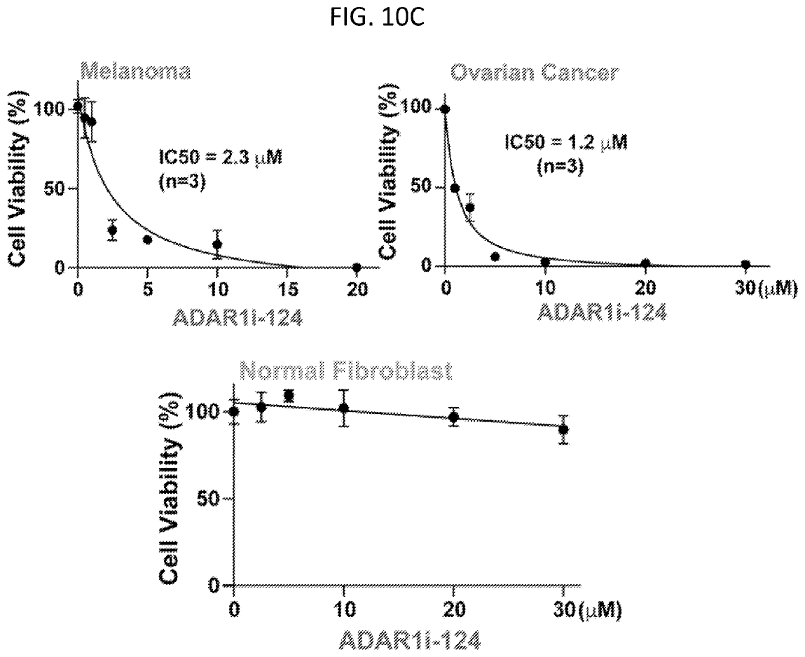

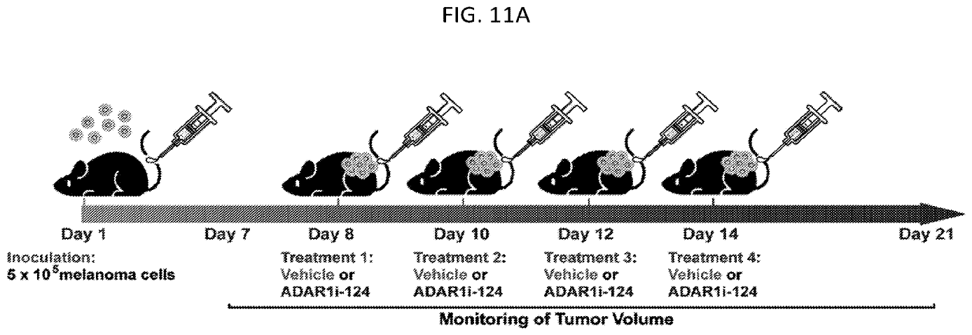

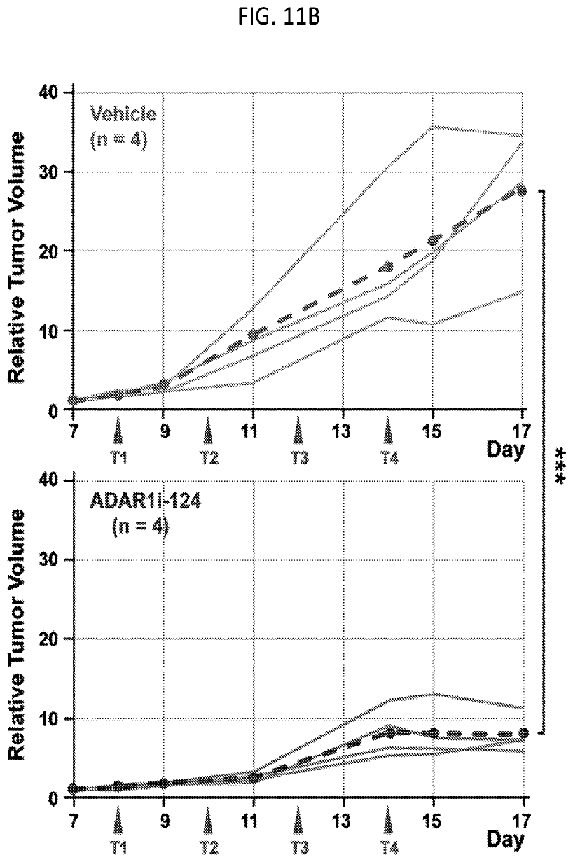

Interestingly, the ratio of ADAR1p150 vs ADARp110 varied significantly among different cancer cell lines (FIG. 10A). Furthermore, ADAR1 knockdown or ADAR1i-124 activated IFN signaling in the cancer cells with elevated ADAR1p150 expression such as YUMM1.7 (FIG. 9B) but not in the cancer cells with elevated ADAR1p110 such as HeLa (not shown), perhaps indicating desensitization of the dsRNA sensing mechanism due to very low expression of MDA5 (22). Finally, ADAR1 knockdown or ADAR1i-124 induced apoptosis in the cancer cells with elevated ADAR1p110 (HeLa cells) and necroptosis in the cancer cells with elevated ADAR1p150 (YUMM1.7 cells) (FIG. 10B). The results indicate that ADAR1i-124 kills cancer cells via different cell death pathways, depending on their relative ADAR1p110 or ADAR1p150 dependency developed in different cancers. Thus, ADAR1i-124 has the potential to act as a “two birds with one stone” cancer therapeutic.

### Example 9. Characterization of Structural Analogs of ADAR1i-124 (Aim 1)

Using our functional screening approach and confirmation in our in vitro editing assay we identified ADAR1i-13 (FIG. 4A). Analysis of 7 ADAR1i-13 analogs led to identification of essential structure-activity relationship (SAR) for ADAR1 inhibition, particularly the presence of the cyano and chloride substituents, and ADAR1i-124 with the best potency for HeLa cell killing with IC50 0.4 μM (FIG. 9A). ADAR1i-124 inhibited both ADAR1p150 and ADAR1p-110, as expected from its binding to the ADAR1 catalytic center pocket (FIG. 5, FIG. 6), whereas ADAR1i-124 did not inhibit ADAR2 (FIG. 4).

Despite the promising properties of ADAR1i-124 for in vitro and in vivo inhibition of ADAR1 activity, we noticed its relatively short t1/2, ~12 hrs, in the culture media determined by loss of its cancer cell killing activity (data not shown). We will modify ADAR1i-124 and derive ADAR1i-124 analogs with higher potency (higher affinity binding and lower IC50) ready to be tested for animal model studies (next section).

Analysis of ADAR1 inhibitory activity in vitro and in cells. ADAR1 RNA editing inhibitory activity of 8 analogs of ADAR1i-124 will be evaluated by in vitro editing assay at varying concentrations of analog compounds as we did for ADAR1i-13 analogs (FIG. 5A). Their IC50 values will be compared to their parental compound ADAR1i-124 (FIG. 4). In vivo inhibition of ADAR1 editing will be monitored by dose-dependent detection of unedited dsRNA-MDA5 complex fibers as we did for ADAR1i-13 analogs (FIG. 7A). RNA-seq analysis will be done to confirm global reduction of ADAR1 site specific A-to-I editing (18).

Analysis of ADAR1i-124 analogs for their selective killing of cancer cells. We will investigate 8 ADAR1i-124 analogs for their dose-dependent selective killing of cancer cells (FIG. 9A) and conduct western blotting analysis to confirm activation of DNA damage, M phase arrest, increased formation of R-loops (RNA:DNA hybrids) (FIG. 8), and induction of apoptosis or necroptosis (FIG. 10), as done with ADAR1i-124. We will monitor cell growth inhibitory IC50 values, since replacement of the hydroxyl methyl group is expected to increase the in vivo stability and consequently lower IC50 values.

SPR analysis of binding affinity of ADAR1i-124 analogs. Binding of ADAR11-124 analogs with in vitro editing inhibitory activity confirmed (above) will be then examined for their specific binding to ADAR1p110-WT but not to ADAR1p110-E912A mutant, as done with ADAR1i-13 and ADAR1i-124 (FIG. 5). We will see whether modification of the hydroxyl methyl group increases their binding. As done for ADAR1i-124 (FIG. 7), docking simulation for fitting of ADAR1i-124 analogs with improved editing inhibitory activity and ADAR1 binding affinity will be done to confirm the mechanistic reason for improvement. Although we expect all ADAR1i-124 analogs to fit into the catalytic center pocket, a collection of ADAR1 mutants (single amino acid directed and truncation) available in our laboratory (42, 56) will be used for the analogs not showing differential binding to ADAR1p110-WT and ADAR1p110-E912A mutant (FIG. 5).

Analysis of ADAR1i-124 analogs for their potency to activate IFN signaling in cancer cells. Inhibition of ADAR1 mediated A-to-I editing of 3′UTR Alu dsRNAs activates oligomerization of unedited dsRNA-MDA5 complexes into fiber structures (FIG. 7A) and induces IFNs in cancer cells, which developed ADAR1p150-dependency (FIG. 10B). This activation of IFN signaling is required to overcome the tumor resistance to PD-1 based immunotherapy (2), one of our motivations to identify ADAR1 inhibitors. ADAR1i-124 upregulated expression of known ISGs (FIG. 9B). Similar qRT-PCR analysis will be done for ADAR1i-124 analogs to evaluate their dose-dependent potency to activate IFN signaling. In addition, we will also conduct RNA-seq analysis to evaluate their effects on global gene expression in addition to ISGs.

Pharmacokinetics analysis of ADAR1i-124 analogs. Finally, ADAR1i-124 and analogs will be evaluated for ADME/PK drug like properties including water solubility and mouse liver microsome stability. Mouse liver microsome stability is a predictive measure of suitable plasma exposure, which is determined by mouse pharmacokinetics analysis. Only compounds with mouse liver microsome stability >20 min will be evaluated in a mouse pharmacokinetic study (IP administration; 50 mg/kg) to evaluate plasma clearance and compound half-life in vivo. Compounds with suitable plasma exposure will be prioritized for in vivo studies.

Statistical considerations. In vitro drug dose-response experiments will be performed independently in triplicate. Analyses of drug dose response with cell viability data will be performed using GraphPad Prism software. IC50 will be estimated using four-parameter logistic regression for each single ADAR1i.

### Example 10: Evaluation of ADAR1 Inhibitors in the Syngeneic Mouse Melanoma Model

ADAR1i-124 induced apoptosis in certain cancer cells such as HeLa by suppressing ADAR1p110 function in control of telomeric repeat R-loops in the nucleus (FIG. 10B). Furthermore, ADAR1i-124 activated type I IFN signaling (FIG. 9B) and induced necroptosis in YUMM1.7 mouse melanoma cells (FIG. 10B). Induction of IFN signaling is expected to contribute to overcoming the resistance to ICB (2). We will test the efficacy of our top selected ADAR1i in vivo in the syngeneic mouse melanoma model. The study will identify the most effective ADAR1i, which could be then prioritized for the next studies in humanized mouse PDX melanoma model.

Toxicity tests and treatments. To study the effect of ADAR1 inhibitors in the context of an intact immune system, tumor cells will be implanted subcutaneously into syngeneic C57BL/6 mice (both male and female). We will first conduct a pilot study to determine the maximum tolerated (MTD) dose of the ADAR1 inhibitor. In fact, we already completed for toxicity tests for ADAR1i-124. A varying amount of ADAR1i-124 in 5% DMSO/10% Solutol/PBS was delivered by intraperitoneal (I.P.) injection. We found that mice well tolerated up to 50 mg/kg for one dose injection and also 5 continuous daily injections. Once the toxicity of each ADAR1i has been tested, we will then assess the effect of a low dose, intermediate dose, and MTD of the selected ADAR1i on inhibiting tumor growth. Mouse melanoma cells (5×10⁵ to 2×10⁶) in Matrigel/PBS will be implanted subcutaneously. Once tumors become palpable (50-100 mm3), mice will be randomized into different treatment groups (vehicle, low dose, intermediate and MTD). Mice will be treated daily via IP injection for 2 weeks and tumor growth and animal survival will be determined. To determine sole effects of ADAR1i, mice will be randomized into two cohorts and treated with either 1) vehicle control, or 2) ADAR1i alone. For combination studies, the effective dose of ADAR1i and lower dose will be tested. In these studies, mice bearing established tumors will be randomized into five different treatment groups: 1) vehicle+control IgG, 2) ADAR1i+control IgG, 3) vehicle+αPD-1 Ab, 4) ADAR1i (low dose)+αPD-1 Ab, 5) ADAR1i (effective dose)+αPD-1 Ab. Combination parameters to be established include optimal doses, schedule, and combination regime. In the first set of experiments, the top selected ADAR1i in 100 μl 5% DMSO/10% Solutol/PBS will be given first to prime the tumor for αPD1 mediated immune response. Unless toxicity issues prevent it, mice will be dosed with ADAR1i throughout the experiment. The αPD-1 antibody (low endotoxin, azide-free, 0.1 EU/μg, purified rat anti-mouse PD-1 antibody, clone RMP1-14, BioLegend) or control rat IgG antibody will be injected via I.P. at a concentration of 10 mg/kg three times per week for two weeks. We will monitor the tumor growth by digital calipers twice a week. Animals will be euthanized on day 21 or when tumors reach 2.0 cm3; endpoints will be tumor volume, and survival. The percent Tumor Growth Inhibition (% TGI) will be calculated and reported for each of the treatment groups (T) versus control (C) using initial (i) and final (f) tumor measurements by the following formula: % TGI=[1−(Tf−Ti/Cf−Ci)]×100.

Analysis of host mice and tumors. Mice will be bled early-on-treatment (EOT), at the time of tumor response (TTR) and at the end of study (EOS). Markers of immune response (58) and levels of circulating tumor cells (CTC) will be assessed. Tumors will be digested using a Tumor Dissociation Kit and gentle MACS Dissociator (Milentyi Biotec). The immune cell infiltration will be assessed by flow cytometry (CD45 positivity). T cell subsets will be defined based on CD3, CD8, and CD4 positivity (87, 88). If strong responses are observed upon treatment, half of the treated mice will be euthanized and the other half will be continuously monitored to determine progression free survival. CTCs (CD45−) will be determined by flow cytometry as previously described (59). Another tumor aliquot will be digested, and myeloid and lymphoid fractions of infiltrating immune cells will be collected using FACS. Cytokine expression in sorted cells will be evaluated by qRT-PCR and intracellular cytokine staining. We will monitor global reduction of ADAR1 mediated A-to-I editing and activation of IFN signaling and ISGs in tumors and host mice by RNA-seq analysis (18).

Further evaluation of ADAR1i tumor killing mechanism. If we observe a positive correlation between changes in certain immune modulating cell populations and tumor suppressive effects, we will perform loss of function studies to determine whether these changes account for or contribute to the observed tumor suppressive effects in the combination treatment. For example, if we observe a correlation with an increase in CD8+ cytotoxic T cells in the combination treatment group compared with either treatment alone, we will deplete CD8+ T cells using an anti-CD8 antibody as we previously published (60). This will allow us to determine whether the depletion of CD8+ T cells is sufficient to block the observed synergy in the combination treatment group. These results will inform us whether ADAR1i-induced changes in the immune modulating cells such as CD8+ T cells in the tumor microenvironment contribute to or account for the observed tumor suppressive effects in the combination treatment group. Direct tumor killing by ADAR1i itself may be tested also in NSG mice to assess relative contributions of immune cells.

### Example 11: Evaluation of ADAR1 Inhibitors in the Humanized Mouse PDX Melanoma Model

Inhibition of the nuclear ADAR1p110 results in accumulation of telomeric variant-repeat R-loops and selective killing of non-ALT (telomerase reactivated) cancer cells (3). It is well known that the telomere length of mice is much longer than that of humans. Thus, ADAR1i may affect mouse and human tumors differently. Furthermore, human and mouse immune systems are significantly different (61, 62). As involvement of innate immunity is anticipated in the ADAR1i mechanism for selective killing of cancers, it is ideal to test the ADAR1i efficacy with human tumors and humanized immunity. To this end, we will test ADAR1i using patient derived xenograft (PDX) melanomas in humanized BLT (bone marrow, liver, thymus) mice (Hu-BLT mice). The HLA matching BLT mice are prepared by Dr. Zhe Yuan (Director of The Humanized Mouse Models of Disease Core), who has the IRB protocol already approved for human subjects (IRB 21809310 Sep. 27, 2022). Dr. Meenhard Herlyn provides a collection of HLA predetermined PDX melanomas cell lines. Human melanomas established in these humanized models generate immune responses and respond to immune therapeutics (51, 52). This part of proposal will be done in collaboration with Drs. Villanueva and Yuan. Dr. Herlyn will also help us with experimental designing and data analysis. They all have extensive experience with humanized mouse and PDX melanoma studies (7, 48-52). The most effective ADAR1i identified through these in vivo studies will be then prioritized for next stage studies that may ultimately lead to testing these ADAR1 inhibitors in future clinical studies.

Human Fetal Tissue Research Approach [in compliance with required added strategy text per NOD-19-128/NOT-OD-19-137/NOT-OD-21-111 for research involving the use of human fetal tissue (HFT)].

Proposed characteristics, procurement, and procedures. Matched human fetal bone marrow, liver, and thymus tissues (HFTs) at gestational week 16-20 will be acquired from Advanced Bioscience Resources, Inc. (ABR). HFT is acquired by ABR under an IRB-approved informed consent vehicle (see informed consent document in Appendix) that is signed by both the woman and the person obtaining consent and includes language acknowledging that 1) informed consent for donation of HFT was obtained by someone other than the person who obtained the informed consent for abortion, 2) occurred after the informed consent for abortion, 3) will not affect the method of abortion and further that 4) no enticements, benefits, or financial incentives were used at any level of the process to incentivize abortion or the donation of HFT (see HFT Compliance Assurance Letter in Appendix). HFT will be overnight shipped from ABR by FedEx and processed immediately at the Wistar Humanized Mouse Models of Disease Core for transplant into NSG mice (BLT mice). Any HFT not transplanted into mice and all BLT mice at end of study will be disposed into the biohazardous waste stream for transport and offsite incineration by a certified commercial provider.

Justification for the use of HFT. In vitro approaches are inadequate to test protection modalities or translational efficacy of in vivo outcomes that include both blood, trafficking, leukocyte extravasation, and tissue-based microenvironments with both CD4 T-cells and myeloid cells. As the ultimate goal of this research is identification of new ADAR1i therapeutics for cancer treatment and improvement of ICB-based immunotherapy efficacy, the scientific approach should be sufficiently stringent to model hurdles inherent to clinical translation, therefore, an in vivo model is required. There is no alternative in vivo model system using human PDX melanoma cells that allows for the study of cancer immunotherapy outside of humanized mice (see expanded rationale for use of HFT in Human Subject Protection Section). Though non-HFT humanized mouse models exist, sufficient T cell engraftment/function (huHSC with adult mobilized CD34) (63), sufficient single donor HSC sources (huHSC with cord blood CD34) (64), are unproven in the field of immunotherapy (e.g. Neothy model) (65), and are thus less suitable than hu-BLT mice for this proposal.

Generation of hu-BLT mice. Experiments in this proposal are designed to determine the efficacy of ADAR1 inhibitors as cancer therapeutics and ICB-based immunotherapy therapeutics in humanized BLT mice (hu-BLT mice). Hu-BLT mice are generated as previously described (49, 50) in accordance with The Wistar Institute Animal Care and Research Committee regulations (protocol #201360 Aug. 17, 2022). Briefly, 6-8 weeks old female NSG (NOD.Cg-Prkdcscid Il2rgtm1 Wjl/SzJ, Wistar) mice are pretreated with busulfan at 30 mg/kg and then implanted with human fetal thymic tissue fragments and fetal liver tissue fragments under the murine renal capsule. Following the surgery, mice are injected via the tail vein with CD34+ hematopoietic stem cells isolated from human fetal liver tissues. Human fetal liver and thymus tissues are procured from Advanced Bioscience Resources (Alameda, CA). Twelve weeks post-surgery, human immune cell reconstitution in peripheral blood is determined by flow cytometry analysis using the following antibodies: mCD45-AF700, hCD45-FITC, hCD3-BUV805, hCD4-BUV395, hCD8-PerCP-Cy5.5 and Fixable Viability Stain 510.

Treatments and analysis. The efficacy of ADAR1i alone or combining with αPD1 Ab for suppression of tumor growth will be assessed as described in the previous section (Aim 2-1). We have at our disposal the largest collection (>500) of clinically and genetically annotated melanoma PDX. ADAR1i-124 killed one PDX melanoma cell line, WM3000, in a dose-dependent manner (FIG. 9A). We will use the human melanoma cell lines/PDXs in subcutaneous tumor assays in the hu-BLT mice. Human melanomas will be established in the hu-BLT mice and randomized into 4 treatment groups (n=5 per group): 1) ADAR1i alone, 2) αPD-1 Ab alone, 3) combination ADAR1i/αPD-1, 4) control IgG. Tumor volumes and overall survival will be determined as described in the previous section (Aim 2-1). Tumors will be biopsied and blood collected at interval periods (at EOT, e.g. 7 days, at TTR, and at EOS) and analyzed for hallmark melanoma features as above. In addition, we will use standard immunohistochemistry and flow cytometry of tumor cell suspensions to define the infiltrating immune cell populations and their relative activation/exhaustion state (66) as described in the previous section.

### Example 12: Evaluation of In Vivo Mouse Study Results

Statistical considerations. For in vivo experiments, mean, standard deviation (SD), median tumor volume and tumor weight will be calculated, and the distribution of data will be examined to determine if data transformation is needed for normal theory methods. ANOVA or Kruskal-Wallis test with post-hoc Bonferroni's adjustment will be performed for data analysis. For survival data analysis, log-rank test will be applied. A two-sided p-value of less than 0.05 will be considered statistically significant.

Power analyses: Based on our previous similar studies, we would like to detect a large effect size of 1.73 in the difference of averaged changes in tumor volume at the end of the experiments between the treatment groups and control group. We estimate that using 10 mice per group will ensure that our study will have 80% power to detect an expected large effect size of 1.73 at a two-sided type I error of 0.008 (Bonferroni's adjustment for 6 multiple comparisons from 4 groups experiments). Here effect size refers to the difference between group means divided by the standard deviation.

Expected outcomes, pitfalls, and alternate future approaches. These studies will determine the therapeutic value of ADAR1i alone and a combination with αPD-1 Ab for melanomas. We anticipate that ADAR1i alone will significantly suppress the growth of telomerase reactivated (or non-ALT) tumors. However, we foresee that in αPD-1 Ab combination studies we will be able to use lower concentrations of each ADAR1i and decrease toxicity. We are also prepared to test different ADAR1i schedules aimed at decreasing possible toxicities.

### REFERENCES

1. Fritzell K, Xu L D, Otrocka M, Andreasson C, Ohman M. Sensitive ADAR editing reporter in cancer cells enables high-throughput screening of small molecule libraries. Nucleic acids research. 2019; 47 (4): e22. Epub 2018 Dec. 28. PubMed PMID: 30590609; PMCID: PMC6393238.
2. Ishizuka J J, Manguso R T, Cheruiyot C K, Bi K, Panda A, Iracheta-Vellve A, Miller B C, Du P P, Yates K B, Dubrot J, Buchumenski I, Comstock D E, Brown F D, Ayer A, Kohnle I C, Pope H W, Zimmer M D, Sen D R, Lane-Reticker S K, Robitschek E J, Griffin G K, Collins N B, Long A H, Doench J G, Kozono D, Levanon E Y, Haining W N. Loss of ADAR1 in tumours overcomes resistance to immune checkpoint blockade. Nature. 2019; 565 (7737): 43-8. Epub 2018 Dec. 19. PubMed PMID: 30559380.
3. Shiromoto Y, Sakurai M, Minakuchi M, Ariyoshi K, Nishikura K. ADAR1 RNA editing enzyme regulates R-loop formation and genome stability at telomeres in cancer cells. Nature communications. 2021; 12 (1): 1654. Epub 2021 Mar. 14. PubMed PMID: 33712600; PMCID: PMC7955049.
4. Nishikura K. Functions and regulation of RNA editing by ADAR deaminases. Annual review of biochemistry. 2010; 79:321-49. PubMed PMID: 20192758; PMCID: 2953425.
5. Nishikura K. A-to-I editing of coding and non-coding RNAs by ADARs. Nature reviews. 2016; 17 (2): 83-96. PubMed PMID: 26648264.
6. Wu S, Fukumoto T, Lin J, Nacarelli T, Wang Y, Ong D, Liu H, Fatkhutdinov N, Zundell J A, Karakashev S, Zhou W, Schwartz L E, Tang H Y, Drapkin R, Liu Q, Huntsman D G, Kossenkov A V, Speicher D W, Schug Z T, Van Dang C, Zhang R. Targeting glutamine dependence through GLS1 inhibition suppresses ARID1A- inactivated clear cell ovarian carcinoma. Nat Cancer. 2021; 2 (2): 189-200. Epub 2021 Jun. 5. PubMed PMID: 34085048; PMCID: PMC8168620.
7. Erkes D A, Field C O, Capparelli C, Tiago M, Purwin T J, Chervoneva I, Berger A C, Hartsough E J, Villanueva J, Aplin A E. The next-generation BET inhibitor, PLX51107, delays melanoma growth in a CD8− mediated manner. Pigment Cell Melanoma Res. 2019; 32 (5): 687-96. Epub 2019 May 8. PubMed PMID: 31063649; PMCID: PMC6697571.
8. Bass B L, Weintraub H. A developmentally regulated activity that unwinds RNA duplexes. Cell. 1987; 48 (4): 607-13. PubMed PMID: 2434241.
9. Rebagliati M R, Melton D A. Antisense RNA injections in fertilized frog eggs reveal an RNA duplex unwinding activity. Cell. 1987; 48 (4): 599-605. PubMed PMID: 2434240.
10. Bass B L, Weintraub H. An unwinding activity that covalently modifies its double-stranded RNA substrate. Cell. 1988; 55 (6): 1089-98. PubMed PMID: 3203381.
11. Wagner R W, Smith J E, Cooperman B S, Nishikura K. A double-stranded RNA unwinding activity introduces structural alterations by means of adenosine to inosine conversions in mammalian cells and Xenopus eggs. Proceedings of the National Academy of Sciences of the United States of America. 1989; 86 (8): 2647-51. PubMed PMID: 2704740.
12. Zheng Y, Lorenzo C, Beal P A. DNA editing in DNA/RNA hybrids by adenosine deaminases that act on RNA. Nucleic acids research. 2017; 45 (6): 3369-77. PubMed PMID: 28132026; PMCID: 5389660.
13. Kim U, Wang Y, Sanford T, Zeng Y, Nishikura K. Molecular cloning of cDNA for double-stranded RNA adenosine deaminase, a candidate enzyme for nuclear RNA editing. Proceedings of the National Academy of Sciences of the United States of America. 1994; 91 (24): 11457-61. PubMed PMID: 7972084.
14. Lai F, Chen C X, Carter K C, Nishikura K. Editing of glutamate receptor B subunit ion channel RNAs by four alternatively spliced DRADA2 double-stranded RNA adenosine deaminases. Molecular and cellular biology. 1997; 17 (5): 2413-24. PubMed PMID: 9111310.
15. Chen C X, Cho D S, Wang Q, Lai F, Carter K C, Nishikura K. A third member of the RNA-specific adenosine deaminase gene family, ADAR3, contains both single- and double-stranded RNA binding domains. RNA. 2000; 6 (5): 755-67. PubMed PMID: 10836796.
16. Hood J L, Emeson R B. Editing of neurotransmitter receptor and ion channel RNAs in the nervous system. Curr Top Microbiol Immunol. 2012; 353:61-90. PubMed PMID: 21796513.
17. Ramaswami G, Lin W, Piskol R, Tan M H, Davis C, Li J B. Accurate identification of human Alu and non-Alu RNA editing sites. Nat Methods. 2012; 9 (6): 579-81. Epub 2012 Apr. 10. PubMed PMID: 22484847.
18. Tan M H, Li Q, Shanmugam R, Piskol R, Kohler J, Young A N, Liu K I, Zhang R, Ramaswami G, Ariyoshi K, Gupte A, Keegan L P, George C X, Ramu A, Huang N, Pollina E A, Leeman D S, Rustighi A, Goh Y P S, Consortium G T, Laboratory D A, Coordinating Center-Analysis Working G, Statistical Methods groups-Analysis Working G, Enhancing Gg, Fund NIHC, Nih/Nci, Nih/Nhgri, Nih/Nimh, Nih/Nida, Biospecimen Collection Source Site N, Biospecimen Collection Source Site R, Biospecimen Core Resource V, Brain Bank Repository-University of Miami Brain Endowment B, Leidos Biomedical-Project M, Study E, Genome Browser Data I, Visualization EBI, Genome Browser Data I, Visualization-Ucsc Genomics Institute UoCSC, Chawla A, Del Sal G, Peltz G, Brunet A, Conrad D F, Samuel C E, O'Connell M A, Walkley C R, Nishikura K, Li J B. Dynamic landscape and regulation of RNA editing in mammals. Nature. 2017; 550 (7675): 249-54. PubMed PMID: 29022589.
19. Hartner J C, Schmittwolf C, Kispert A, Muller A M, Higuchi M, Seeburg P H. Liver disintegration in the mouse embryo caused by deficiency in the RNA-editing enzyme ADAR1. The Journal of biological chemistry. 2004; 279 (6): 4894-902. PubMed PMID: 14615479.
20. Wang Q, Miyakoda M, Yang W, Khillan J, Stachura D L, Weiss M J, Nishikura K. Stress-induced apoptosis associated with null mutation of ADAR1 RNA editing deaminase gene. The Journal of biological chemistry. 2004; 279 (6): 4952-61. PubMed PMID: 14613934.
21. Liddicoat B J, Piskol R, Chalk A M, Ramaswami G, Higuchi M, Hartner J C, Li J B, Seeburg P H, Walkley C R. RNA editing by ADAR1 prevents MDA5 sensing of endogenous dsRNA as nonself. Science. 2015; 349 (6252): 1115-20. PubMed PMID: 26275108.
22. Mannion N M, Greenwood S M, Young R, Cox S, Brindle J, Read D, Nellaker C, Vesely C, Ponting C P, McLaughlin P J, Jantsch M F, Dorin J, Adams I R, Scadden A D, Ohman M, Keegan L P, O'Connell M A. The RNA-Editing Enzyme ADAR1 Controls Innate Immune Responses to RNA. Cell reports. 2014; 9 (4): 1482-94. PubMed PMID: 25456137.
23. Pestal K, Funk C C, Snyder J M, Price N D, Treuting P M, Stetson D B. Isoforms of RNA-Editing Enzyme ADAR1 Independently Control Nucleic Acid Sensor MDA5-Driven Autoimmunity and Multi-organ Development. Immunity. 2015; 43 (5): 933-44. PubMed PMID: 26588779; PMCID: 4654992.
24. Niehrs C, Luke B. Regulatory R-loops as facilitators of gene expression and genome stability. Nature reviews. 2020. Epub 2020 Feb. 2. PubMed PMID: 32005969.
25. Santos-Pereira J M, Aguilera A. R loops: new modulators of genome dynamics and function. Nature reviews. 2015; 16 (10): 583-97. PubMed PMID: 26370899.
26. Blasco M A. Telomeres and human disease: ageing, cancer and beyond. Nature reviews. 2005; 6 (8): 611-22. Epub 2005 Sep. 2. PubMed PMID: 16136653.
27. de Lange T. Shelterin-Mediated Telomere Protection. Annu Rev Genet. 2018; 52:223-47. Epub 2018 Sep. 13. PubMed PMID: 30208292.
28. Lee Y W, Arora R, Wischnewski H, Azzalin C M. TRF1 participates in chromosome end protection by averting TRF2-dependent telomeric R loops. Nature structural & molecular biology. 2018; 25 (2): 147-53. Epub 2018 Jan. 24. PubMed PMID: 29358759; PMCID: PMC5808845.
29. Garcia-Muse T, Aguilera A. R Loops: From Physiological to Pathological Roles. Cell. 2019; 179 (3): 604-18. Epub 2019 Oct. 15. PubMed PMID: 31607512.
30. Greenberg R A. Telomeres, crisis and cancer. Curr Mol Med. 2005; 5 (2): 213-8. Epub 2005 Jun. 25. PubMed PMID: 15974875.
31. Zipeto M A, Court A C, Sadarangani A, Delos Santos N P, Balaian L, Chun H J, Pineda G, Morris S R, Mason C N, Geron I, Barrett C, Goff D J, Wall R, Pellecchia M, Minden M, Frazer K A, Marra M A, Crews L A, Jiang Q, Jamieson C H M. ADAR1 Activation Drives Leukemia Stem Cell Self-Renewal by Impairing Let-7 Biogenesis. Cell Stem Cell. 2016; 19 (2): 177-91. Epub 2016 Jun. 14. PubMed PMID: 27292188; PMCID: PMC4975616.
32. Ramirez-Moya J, Baker A R, Slack F J, Santisteban P. ADAR1-mediated RNA editing is a novel oncogenic process in thyroid cancer and regulates miR-200 activity. Oncogene. 2020; 39 (18): 3738-53. Epub 2020 Mar. 12. PubMed PMID: 32157211; PMCID: PMC7190574.
33. Ding H Y, Yang W Y, Zhang L H, Li L, Xie F, Li H Y, Chen X Y, Tu Z, Li Y, Chen Y, Yang S Y. 8-Chloro-Adenosine Inhibits Proliferation of MDA-MB-231 and SK-BR-3 Breast Cancer Cells by Regulating ADAR1/p53 Signaling Pathway. Cell Transplant. 2020; 29:963689720958656. Epub 2020 Sep. 11. PubMed PMID: 32907379; PMCID: PMC7784596.
34. Kearney A Y, Fan Y H, Giri U, Saigal B, Gandhi V, Heymach J V, Zurita A J. 8-Chloroadenosine Sensitivity in Renal Cell Carcinoma Is Associated with AMPK Activation and mTOR Pathway Inhibition. PloS one. 2015; 10 (8): e0135962. Epub 2015 Aug. 28. PubMed PMID: 26313261; PMCID: PMC4552467.
35. Cottrell K A, Torres L S, Dizon M G, Weber J D. 8-azaadenosine and 8-chloroadenosine are not selective inhibitors of ADAR. Cancer Res Commun. 2021; 1 (2): 56-64. Epub 2022 May 20. PubMed PMID: 35586115; PMCID: PMC9113518.
36. Arora R, Lee Y, Wischnewski H, Brun C M, Schwarz T, Azzalin C M. RNaseH1 regulates TERRA-telomeric DNA hybrids and telomere maintenance in ALT tumour cells. Nature communications. 2014; 5:5220. Epub 2014 Oct. 22. PubMed PMID: 25330849; PMCID: PMC4218956.
37. Sakurai M, Shiromoto Y, Ota H, Song C, Kossenkov A V, Wickramasinghe J, Showe L C, Skordalakes E, Tang H Y, Speicher D W, Nishikura K. ADAR1 controls apoptosis of stressed cells by inhibiting Staufen1-mediated mRNA decay. Nature structural & molecular biology. 2017; 24 (6): 534-43. PubMed PMID: 28436945; PMCID: 5461201.
38. Hao X, Shiromoto Y, Sakurai M, Towers M, Zhang Q, Wu S, Havas A, Wang L, Berger S, Adams P D, Tian B, Nishikura K, Kossenkov A V, Liu P, Zhang R. ADAR1 downregulation by autophagy drives senescence independently of RNA editing by enhancing p16(INK4a) levels. Nat Cell Biol. 2022; 24 (8): 1202-10. Epub 2022 Jul. 20. PubMed PMID: 35851616; PMCID: PMC9757154.
39. Shiromoto Y, Sakurai M, Qu H, Kossenkov A V, Nishikura K. Processing of Alu small RNAs by DICER/ADAR1 complexes and their RNAi targets. RNA. 2020; 26 (12): 1801-14. Epub 2020 Aug. 21. PubMed PMID: 32817447; PMCID: PMC7668262.
40. Boguslawski S J, Smith D E, Michalak M A, Mickelson K E, Yehle C O, Patterson W L, Carrico R J. Characterization of monoclonal antibody to DNA.RNA and its application to immunodetection of hybrids. J Immunol Methods. 1986; 89 (1): 123-30. PubMed PMID: 2422282.
41. Conomos D, Stutz M D, Hills M, Neumann A A, Bryan T M, Reddel R R, Pickett H A. Variant repeats are interspersed throughout the telomeres and recruit nuclear receptors in ALT cells. J Cell Biol. 2012; 199 (6): 893-906. Epub 2012 Dec. 12. PubMed PMID: 23229897; PMCID: PMC3518223.
42. Lai F, Drakas R, Nishikura K. Mutagenic analysis of double-stranded RNA adenosine deaminase, a candidate enzyme for RNA editing of glutamate-gated ion channel transcripts. The Journal of biological chemistry. 1995; 270 (29): 17098-105. PubMed PMID: 7615504.
43. Macbeth M R, Schubert H L, Vandemark A P, Lingam A T, Hill C P, Bass B L. Inositol hexakisphosphate is bound in the ADAR2 core and required for RNA editing. Science. 2005; 309 (5740): 1534-9. PubMed PMID: 16141067.
44. del Toro Duany Y, Wu B, Hur S. MDA5-filament, dynamics and disease. Curr Opin Virol. 2015; 12:20-5. Epub 2015 Feb. 14. PubMed PMID: 25676875; PMCID: PMC4470736.
45. Tikhmyanova N, Tutton S, Martin K A, Lu F, Kossenkov A V, Paparoidamis N, Kenney S, Salvino J M, Lieberman P M. Small molecule perturbation of the CAND1-Cullin1-ubiquitin cycle stabilizes p53 and triggers Epstein-Barr virus reactivation. PLOS Pathog. 2017; 13 (7): e1006517. Epub 2017 Jul. 18. PubMed PMID: 28715492; PMCID: PMC5531659.
46. Salvino J M, Srikanth Y V V, Lou R, Oyer H M, Chen N, Kim F J. Novel small molecule guanidine Sigmal inhibitors for advanced prostate cancer. Bioorg Med Chem Lett. 2017; 27 (10): 2216-20. Epub 2017 Apr. 8. PubMed PMID: 28385503; PMCID: PMC5714280.
47. Kelich J, Aramburu T, van der Vis J J, Showe L, Kossenkov A, van der Smagt J, Massink M, Schoemaker A, Hennekam E, Veltkamp M, van Moorsel C H M, Skordalakes E. Telomere dysfunction implicates POT1 in patients with idiopathic pulmonary fibrosis. J Exp Med. 2022; 219 (5). Epub 2022 Apr. 15. PubMed PMID: 35420632; PMCID: PMC9014792.
48. Wang B, Zhang X L, Li C X, Liu N N, Hu M, Gong Z C. ANLN promotes carcinogenesis in oral cancer by regulating the PI3K/mTOR signaling pathway. Head Face Med. 2021; 17 (1): 18. Epub 2021 Jun. 5. PubMed PMID: 34082790; PMCID: PMC8173900.
49. Yuan Z, Kang G, Daharsh L, Fan W, Li Q. SIVcpz closely related to the ancestral HIV-1 is less or non-pathogenic to humans in a hu-BLT mouse model. Emerg Microbes Infect. 2018; 7 (1): 59. Epub 2018 Apr. 5. PubMed PMID: 29615603; PMCID: PMC5882851.
50. Yuan Z, Giron L B, Hart C, Gyampoh A, Koshy J, Hong K Y, Niki T, Premeaux T A, Ndhlovu L C, Deleage C, Montaner L J, Abdel-Mohsen M. Human galectin-9 promotes the expansion of HIV reservoirs in vivo in humanized mice. AIDS. 2022. Epub 2022 Dec. 3. PubMed PMID: 36460646.
51. Somasundaram R, Connelly T, Choi R, Choi H, Samarkina A, Li L, Gregorio E, Chen Y, Thakur R, Abdel-Mohsen M, Beqiri M, Kiernan M, Perego M, Wang F, Xiao M, Brafford P, Yang X, Xu X, Secreto A, Danet-Desnoyers G, Traum D, Kaestner K H, Huang A C, Hristova D, Wang J, Fukunaga-Kalabis M, Krepler C, Ping-Chen F, Zhou X, Gutierrez A, Rebecca V W, Vonteddu P, Dotiwala F, Bala S, Majumdar S, Dweep H, Wickramasinghe J, Kossenkov A V, Reyes-Arbujas J, Santiago K, Nguyen T, Griss J, Keeney F, Hayden J, Gavin B J, Weiner D, Montaner L J, Liu Q, Peiffer L, Becker J, Burton E M, Davies M A, Tetzlaff M T, Muthumani K, Wargo J A, Gabrilovich D, Herlyn M. Tumor-infiltrating mast cells are associated with resistance to anti-PD-1 therapy. Nature communications. 2021; 12 (1): 346. Epub 2021 Jan. 14. PubMed PMID: 33436641; PMCID: PMC7804257.
52. Gregorio E, Ramirez-Salazar E, DeRosa K, Choi R, Li L, Herlyn M. Production of Humanized Mouse via Thymic Renal Capsule Grafting, CD34+ Cells Injection, and Cytokine Delivery. J Vis Exp. 2021 (175). Epub 2021 Oct. 12. PubMed PMID: 34633384.
53. Garman B, Anastopoulos I N, Krepler C, Brafford P, Sproesser K, Jiang Y, Wubbenhorst B, Amaravadi R, Bennett J, Beqiri M, Elder D, Flaherty K T, Frederick D T, Gangadhar T C, Guarino M, Hoon D, Karakousis G, Liu Q, Mitra N, Petrelli N J, Schuchter L, Shannan B, Shields C L, Wargo J, Wenz B, Wilson M A, Xiao M, Xu W, Xu X, Yin X, Zhang N R, Davies M A, Herlyn M, Nathanson K L. Genetic and Genomic Characterization of 462 Melanoma Patient-Derived Xenografts, Tumor Biopsies, and Cell Lines. Cell reports. 2017; 21 (7): 1936-52. Epub 2017 Nov. 16. PubMed PMID: 29141224; PMCID: PMC5709812.
54. Kung C P, Liu Q, Murphy M E. The codon 72 polymorphism of p53 influences cell fate following nutrient deprivation. Cancer Biol Ther. 2017; 18 (7): 484-91. Epub 2017 May 6. PubMed PMID: 28475405; PMCID: PMC5639853.
55. Nishikura K. Editor meets silencer: crosstalk between RNA editing and RNA interference. Nature reviews. 2006; 7 (12): 919-31. PubMed PMID: 17139332.
56. Ota H, Sakurai M, Gupta R, Valente L, Wulff B-E, Ariyoshi K, lizasa H, Davuluri R V, Nishikura K. ADAR1 forms a complex with Dicer to promote microRNA processing and RNA-induced gene silencing. Cell. 2013; 152:575-89. PubMed PMID: 23622242; PMCID: 3651894.
57. Miller K D, Pniewski K, Perry C E, Papp S B, Shaffer J D, Velasco-Silva J N, Casciano J C, Aramburu T M, Srikanth Y V V, Cassel J, Skordalakes E, Kossenkov A V, Salvino J M, Schug Z T. Targeting ACSS2 with a Transition-State Mimetic Inhibits Triple-Negative Breast Cancer Growth. Cancer Res. 2021; 81 (5): 1252-64. Epub 2021 Jan. 9. PubMed PMID: 33414169; PMCID: PMC8026699.
58. Li H, van der Merwe P A, Sivakumar S. Biomarkers of response to PD-1 pathway blockade. Br J Cancer. 2022; 126 (12): 1663-75. Epub 2022 Mar. 2. PubMed PMID: 35228677.
59. Reyes-Uribe P, Adrianzen-Ruesta M P, Deng Z, Echevarria-Vargas I, Mender I, Saheb S, Liu Q, Altieri D C, Murphy M E, Shay J W, Lieberman P M, Villanueva J. Exploiting TERT dependency as a therapeutic strategy for NRAS-mutant melanoma. Oncogene. 2018; 37 (30): 4058-72. Epub 2018 Apr. 27. PubMed PMID: 29695835; PMCID: PMC6062502.
60. Zhu H, Bengsch F, Svoronos N, Rutkowski M R, Bitler B G, Allegrezza M J, Yokoyama Y, Kossenkov A V, Bradner J E, Conejo-Garcia J R, Zhang R. BET Bromodomain Inhibition Promotes Anti-tumor Immunity by Suppressing PD-L1 Expression. Cell reports. 2016; 16 (11): 2829-37. Epub 2016 Sep. 15. PubMed PMID: 27626654; PMCID: PMC5177024.
61. Shay T, Jojic V, Zuk O, Rothamel K, Puyraimond-Zemmour D, Feng T, Wakamatsu E, Benoist C, Koller D, Regev A, ImmGen C. Conservation and divergence in the transcriptional programs of the human and mouse immune systems. Proceedings of the National Academy of Sciences of the United States of America. 2013; 110 (8): 2946-51. Epub 2013 Feb. 6. PubMed PMID: 23382184; PMCID: PMC3581886.
62. Bjornson-Hooper Z B, Fragiadakis G K, Spitzer M H, Chen H, Madhireddy D, Hu K, Lundsten K, McIlwain D R, Nolan G P. A Comprehensive Atlas of Immunological Differences Between Humans, Mice, and Non-Human Primates. Front Immunol. 2022; 13:867015. Epub 2022 Apr. 2. PubMed PMID: 35359965; PMCID: PMC8962947.
63. Lepus C M, Gibson T F, Gerber S A, Kawikova I, Szczepanik M, Hossain J, Ablamunits V, Kirkiles-Smith N, Herold K C, Donis R O, Bothwell A L, Pober J S, Harding M J. Comparison of human fetal liver, umbilical cord blood, and adult blood hematopoietic stem cell engraftment in NOD-scid/gammac−/−, Balb/c-Rag1−/− gammac−/−, and C.B-17-scid/bg immunodeficient mice. Hum Immunol. 2009; 70 (10): 790-802. Epub 2009 Jun. 16. PubMed PMID: 19524633; PMCID: PMC2949440.
64. Jaime-Perez J C, Monreal-Robles R, Rodriguez-Romo L N, Mancias-Guerra C, Herrera-Garza J L, Gomez-Almaguer D. Evaluation of volume and total nucleated cell count as cord blood selection parameters: a receiver operating characteristic curve modeling approach. Am J Clin Pathol. 2011; 136 (5): 721-6. Epub 2011 Oct. 28. PubMed PMID: 22031310.
65. Brown M E, Zhou Y, McIntosh B E, Norman I G, Lou H E, Biermann M, Sullivan J A, Kamp T J, Thomson J A, Anagnostopoulos P V, Burlingham W J. A Humanized Mouse Model Generated Using Surplus Neonatal Tissue. Stem Cell Reports. 2018; 10 (4): 1175-83. Epub 2018 Mar. 27. PubMed PMID: 29576539; PMCID: PMC5998340.
66. Natale C A, Li J, Pitarresi J R, Norgard R J, Dentchev T, Capell B C, Seykora J T, Stanger B Z, Ridky T W. Pharmacologic Activation of the G Protein-Coupled Estrogen Receptor Inhibits Pancreatic Ductal Adenocarcinoma. Cell Mol Gastroenterol Hepatol. 2020; 10 (4): 868-80 el. Epub 2020 May 8. PubMed PMID: 32376419; PMCID: PMC7578406.
67. Tenora L, Alt J, Dash R P, Gadiano A J, Novotna K, Veeravalli V, Lam J, Kirkpatrick Q R, Lemberg K M, Majer P, Rais R, Slusher B S. Tumor-Targeted Delivery of 6-Diazo-5-oxo-1-norleucine (DON) Using Substituted Acetylated Lysine Prodrugs. J Med Chem. 2019; 62 (7): 3524-38. Epub 2019 Mar. 21. PubMed PMID: 30892035; PMCID: PMC8025739.
68. Low P S, Henne W A, Doorneweerd D D. Discovery and development of folic-acid-based receptor targeting for imaging and therapy of cancer and inflammatory diseases. Acc Chem Res. 2008; 41 (1): 120-9. Epub 2007 Jul. 28. PubMed PMID: 17655275.
69. Yang J, Vlashi E, Low P. Folate-linked drugs for the treatment of cancer and inflammatory diseases. Subcell Biochem. 2012; 56:163-79. Epub 2011 Nov. 26. PubMed PMID: 22116699.
70. Landis S C, Amara S G, Asadullah K, Austin C P, Blumenstein R, Bradley E W, Crystal R G, Darnell R B, Ferrante R J, Fillit H, Finkelstein R, Fisher M, Gendelman H E, Golub R M, Goudreau J L, Gross R A, Gubitz A K, Hesterlee S E, Howells D W, Huguenard J, Kelner K, Koroshetz W, Krainc D, Lazic S E, Levine M S, Macleod M R, McCall J M, Moxley R T, 3rd, Narasimhan K, Noble L J, Perrin S, Porter J D, Steward O, Unger E, Utz U, Silberberg S D. A call for transparent reporting to optimize the predictive value of preclinical research. Nature. 2012; 490 (7419): 187-91. Epub 2012 Oct. 13. PubMed PMID: 23060188; PMCID: PMC3511845.

What is claimed is:

1. A pharmaceutical composition comprising a pharmaceutically acceptable carrier and one or more of:
a) ADARi-13;
b) ADARi-120;
c) ADARi-121;
d) ADARi-122;
e) ADARi-123;
f) ADARi-124;
g) ADARi-125; and
h) ADARi-126,
or a prodrug, derivative, pharmaceutical salt, or analog thereof.

2. The pharmaceutical composition according to claim 1, further comprising a checkpoint inhibitor.

3. The pharmaceutical composition according to claim 2, wherein the checkpoint inhibitor is a PD-1 or PD-L1 inhibitor.

4. The pharmaceutical composition according to any one of claims 1 to 3, further comprising a chemotherapeutic agent.

5. A method of treating cancer comprising administering a pharmaceutical composition comprising a pharmaceutically acceptable carrier and one or more of:
i) ADARi-13;
j) ADARi-120;
k) ADARi-121;
l) ADARi-122;
m) ADARi-123;
n) ADARi-124;
o) ADARi-125; and
p) ADARi-126,
or a prodrug, derivative, pharmaceutical salt, or analog thereof.

6. The method according to claim 5, wherein the method further includes administering a checkpoint inhibitor.

7. The method according to claim 6 or 7, wherein the cancer is a telomerase-reactivated cancer.

8. The method according to claim 7, wherein the cancer is a non-ALT cancer.

9. The method according to any one of claims 5 to 8, wherein the compositions are administered intravenously.
